(19) United States
(12) Patent Application Publication
(10) Pub. No.: US 2008/0004286 A1
(43) Pub. Date: Jan. 3, 2008

(54) Method of Using Substituted Piperidines that Increase P53 Activity

(75) Inventors: Yaolin Wang, Edison, NJ (US); Rumin Zhang, Edison, NJ (US); Yao Ma, Westwood, MA (US); Brian Robert Lahue, Millbury, MA (US); Gerald W. Shipps, Stoneham, MA (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 11/769,003

(22) Filed: Jun. 27, 2007

Related U.S. Application Data
(60) Provisional application No. 60/818,128, filed on Jun. 30, 2006.

(51) Int. Cl. A61K 31/496; A61K 31/4545; A61K 31/454

(52) U.S. Cl. 514/253.09; 514/253.13; 514/318; 514/253.12; 514/316; 514/317

(57) ABSTRACT

The present invention discloses a method of using compounds, which have HDM2 protein antagonist activity, to treat or prevent cancer, other diseases caused by abnormal cell proliferation, diseases associated with HDM2, or diseases caused by inadequate P53 activity.

### FIELD OF THE INVENTION

The present invention relates to the use of compounds as Human Double Minute 2 (“HDM2”) protein inhibitors, regulators or modulators, the use of pharmaceutical compositions containing the compounds and methods of treatment using the compounds and compositions to treat diseases such as, for example, cancer, diseases involving abnormal cell proliferation, diseases associated with HDM2 or diseases associated with inadequate P53 activity. This application claims priority from U.S. provisional application Ser. No. 60/818128 filed Jun. 30, 2006.

### BACKGROUND OF THE INVENTION

The tumor suppressor protein P53 plays a central role in maintaining the integrity of the genome in a cell by regulating the expression of a diverse array of genes responsible for DNA repair, cell cycle and growth arrest, and apoptosis [May et al., Oncogene 18 (53) (1999) p. 7621-7636; Oren, Cell Death Differ. 10 (4) (2003) p. 431-442, Hall and Peters, Adv. Cancer Res., 68: (1996) p. 67-108; Hainaut et al., Nucleic Acid Res., 25: (1997) p. 151-157; Sherr, Cancer Res., 60: (2000) p. 3689-95]. In response to oncogenic stress signals, the cell triggers the P53 transcription factor to activate genes implicated in the regulation cell cycle, which thereby initiates either apoptosis or cell cycle arrest. Apoptosis facilitates the elimination of damaged cells from the organism, while cell cycle arrest enables damaged cells to repair genetic damage [reviewed in Ko et al., Genes & Devel. 10: (1996) p. 1054-1072; Levine, Cell 88: (1997) p. 323-331]. The loss of the safeguard functions of P53 predisposes damaged cells to progress to a cancerous state. Inactivating P53 in mice consistently leads to an unusually high rate of tumors [Donehower et al., Nature, 356, (1992) p. 215-221].

The P53 transcription factor promotes the expression of a number of cell cycle regulatory genes, including its own negative regulator, the gene encoding the Mouse Double Minute 2 (Mdm2) protein [Chene, Nature Reviews Cancer 3: (2003) p. 102-109; Momand, Gene 242 (1-2): (2000) p. 15-29; Zheleva et al. Mini. Rev. Med. Chem. 3 (3): (2003) p. 257-270]. The Mdm2 protein (designated HDM2 in humans) acts to down-regulate P53 activity in an auto-regulatory manner [Wu et al, Genes Dev., 7: (1993) p. 1126-1132; Bairak et al., EMBO J, 12: (1993) p. 461-468]. In the absence of oncogenic stress signals, i.e., under normal cellular conditions, the Mdm2 protein serves to maintain P53 activity at low levels [Wu et al, Genes Dev., 7: (1993) p. 1126-1132; Barak et al., EMBO J, 12: (1993) p. 461-468]. However, in response to cellular DNA damage or under cellular stress, P53 activity increases helping to prevent the propagation of permanently damaged clones of cells by induction of cell cycle and growth arrest or apoptosis.

The regulation of P53 function relies on an appropriate balance between the two components of this P53-Mdm2 auto-regulatory system. Indeed, this balance appears to be essential for cell survival. There are at least three ways that Mdm2 acts to down-regulate P53 activity. First, Mdm2 can bind to the N-terminal transcriptional activation domain of P53 to block expression of P53-responsive genes [Kussie et al., Science, 274: (1996) p. 948-953; Oliner et al., Nature, 362: (1993) p. 857-860; Momand et al, Cell, 69: (1992) p. 1237-1245]. Second, Mdm2 shuttles P53 from the nucleus to the cytoplasm to facilitate the proteolytic degradation of P53 [Roth et al, EMBO J, 17: (1998) p. 554-564; Freedman et al., Mol Cell Biol, 18: (1998) p. 7288-7293; Tao and Levine, Proc. Natl. Acad. Sci. 96: (1999) p. 3077-3080]. Finally, Mdm2 possesses an intrinsic E3 ligase activity for conjugating ubiquitin to P53 for degradation within the ubiquitin-dependent 26S proteosome pathway [Honda et al., FEBS Lett, 420: (1997) p. 25-27; Yasuda, Oncogene 19: (2000) p. 1473-1476]. Thus, Mdm2 impedes the ability of the P53 transcription factor to promote the expression of its target genes by binding P53 in the nucleus. Attenuating the P53-Mdm2 auto-regulatory system can have a critical effect on cell homeostasis. Consistently, a correlation between the overexpression of Mdm2 and tumor formation has been reported [Chene, Nature 3: (2003) p. 102-109]. Functional inactivation of wild type P53 is found in many types of human tumors. Restoring the function of P53 in tumor cells by anti-MDM2 therapy would result in slowing the tumor proliferation and instead stimulate apoptosis. Not surprisingly then, there is currently a substantial effort being made to identify new anticancer agents that hinder the ability of HDM2 to interact with P53 [Chene, Nature 3: (2003) p. 102-109]. Antibodies, peptides, and antisense oligonucleotides have been demonstrated to destroy the P53-Mdm2 interaction, which would release P53 from the negative control of Mdm2, leading to activation of the P53 pathway allowing the normal signals of growth arrest and/or apoptosis to function, which offers a potential therapeutic approach to treating cancer and other diseases characterized by abnormal cell proliferation. [See, e.g., Blaydes et al., Oncogene 14: (1997) p. 1859-1868; Bottger et al., Oncogene 13 (10): (1996) p. 2141-2147].

U.S. Pub. No. 200510037383 A1 describes modified soluble HDM2 protein, nucleic acids that code for this HDM2 protein, the crystals of this protein that are suitable for X-ray crystallization analysis, the use of the proteins and crystals to identify, select, or design compounds that may be used as anticancer agents, and some of the compounds themselves that bind to modified HDM2. (Schering-Plough Corp.).

Small molecules, said to antagonize the P53-Mdm2 interaction, have been described. WO 00/15657 (Zeneca Limited) describes piprizine-4-phenyl derivatives as inhibitors of the interaction between Mdm2 and P53. Grasberger et al. (J. Med. Chem., 48 (2005) p. 909-912) (Johnson & Johnson Pharmaceutical Research & Development L.L. C.) describes discovery and cocrystal structure of benzodiazepinedione as HDM2 antagonists that activate P53 in cells. Galatin et al. (J. Med. Chem. 47 (2004) p 4163-4165) describes a nonpeptidic sulfonamide inhibitor of the P53-Mdm2 interaction and activator of P53 dependent transcription in mdm2-overexpressing cells.

Vassilev (J. Med. Chem. (Perspective) Vol. 48 No. 14, (2005) p. 1-8) (Hoffmann-LaRoche Inc.) describes several small molecule P53 activators as an application in oncology, including the following formulas:

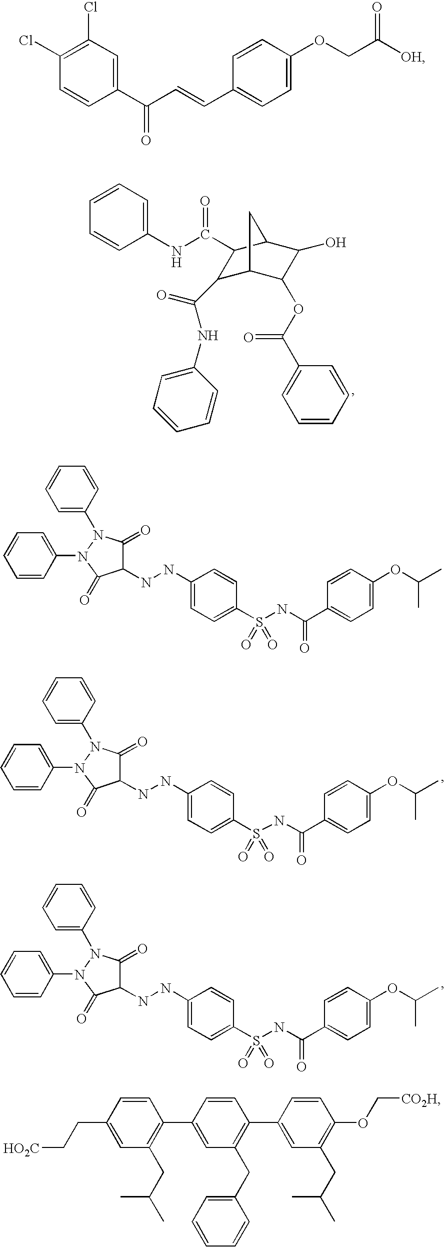

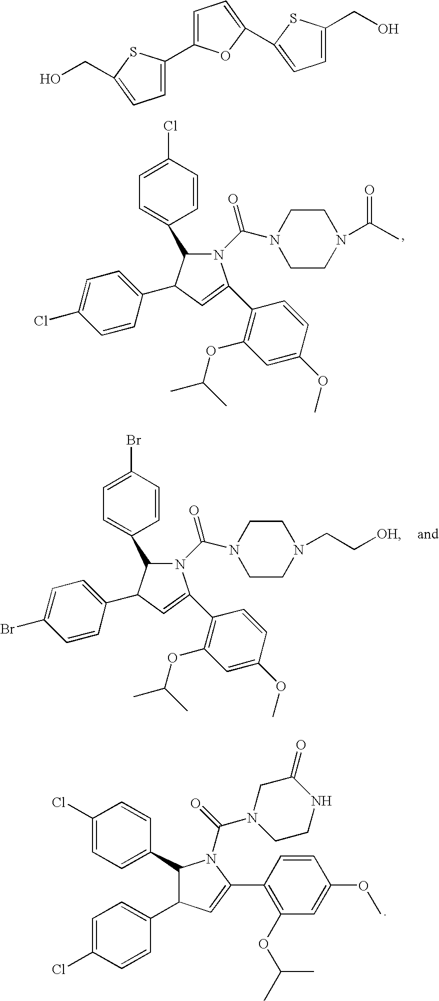

The first four compounds listed above were also described in Totouhi et al. (Current Topics in Medicinal Chemistry Vol. 3, No. 2 (2005) p. 159-166, at 161) (Hoffmann La Roche Inc.). The last three compounds listed above were also described in Vassilev et al. (Science Vol. 303 (2004): p. 844-848) (Hoffmann La Roche Inc.) and their implications on leukemia activity were investigated in Kojima et al. (Blood, Vol. 108 No. 9 (November 2005) p. 3150-3159).

Ding et. al. (J. Am. Chem. Soc. Vol. 127 (2005): 10130-10131) and (J. Med. Chem. Vol. 49 (2006): 3432-3435) describes several spiro-oxindole compounds as Mdm2-P53 inhibitors.

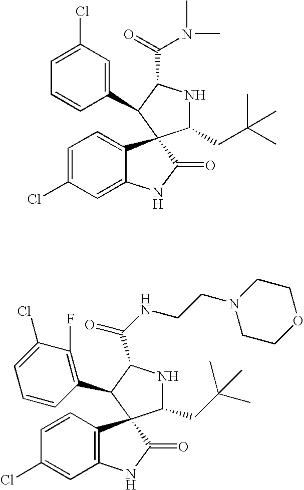

Lu, et. al. (J. Med. Chem. Vol. 49 (2006): 3759-3762) described 7-[anilino(phenyl)methyl]-2-methyl-8-quinolinol as a small molecule inhibitor of MDM2-P53 interaction.

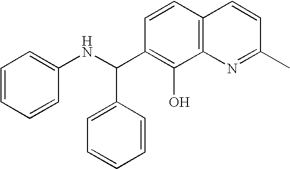

Chène (Molecular Cancer Research Vol. 2: (January 2006) p. 20-28) describes inhibition of the P53-Mdm2 interaction by targeting the protein-protein interface. U.S. Pub. No. 2004/0259867 A1 and 2004/0259884 A1 describes Cis-imidazoles (Hoffmann La Roche Inc.) and WO2005/110996A1 and WO 03/051359 describes Cis-Imidazolines (Hoffmann La Roche Inc.) as compounds that inhibit the interaction of Mdm2 with P53-like peptides resulting in antiproliferation. WO 2004/080460 A1 describes substituted piperidine compounds as Mdm2-P53 inhibitors for treating cancer (Hoffmann La Roche Inc.). EP 0947494 A1 describes phenoxy acetic acid derivatives and phenoxy methyltetrazole that act as antagonists of Mdm2 and interfere with the protein-protein interaction between Mdm2 and P53, which results in anti-tumor properties (Hoffmann La Roche Inc.). Duncan et al., J. Am. Chem. Soc. 123 (4): (2001) p. 554-560 describes a p-53-Mdm2 antagonist, chlorofusin, from a Fusarium Sp. Stoll et al., Biochemistry 40 (2) (2001) p. 336-344 describes chalcone derivatives that antagonize interactions between the human oncoprotein Mdm2 and P53.

There is a need for effective inhibitors of the HDM2 or MDM2 protein in order to treat or prevent cancer, other disease states associated with cell proliferation, diseases associated with HDM2, or diseases caused by inadequate P53 activity. The present application discloses compounds that have potency in inhibiting or antagonizing the HDM2-P53 and Mdm2-P53 interaction and/or activating P53 proteins in cells. The HDM2-P53 and Mdm2-P53 inhibitory activity of such compounds have not been disclosed previously.

### SUMMARY OF THE INVENTION

The present invention provides a method of inhibiting HDM2 protein comprising administering a therapeutically effective amount of at least one compound of the following chemical structure:

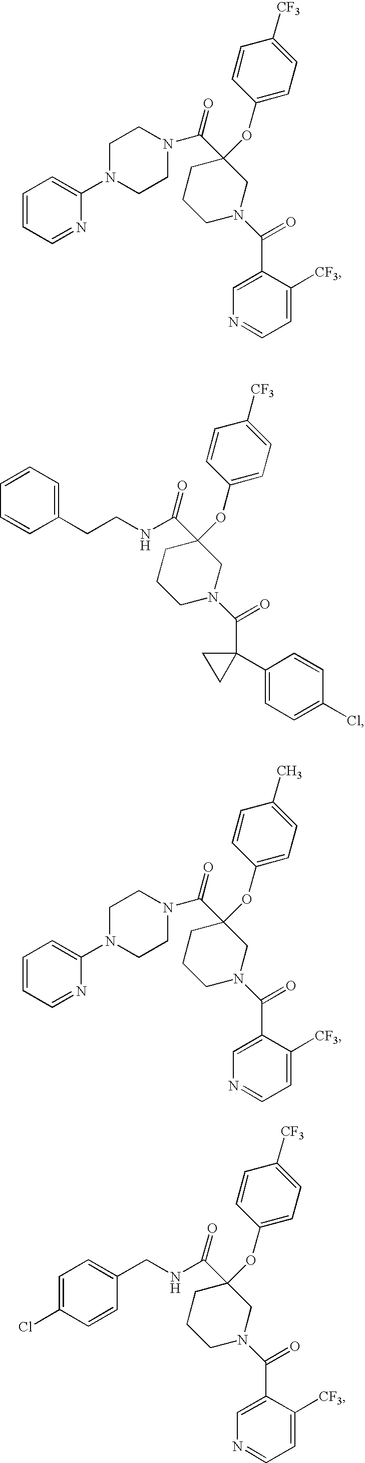

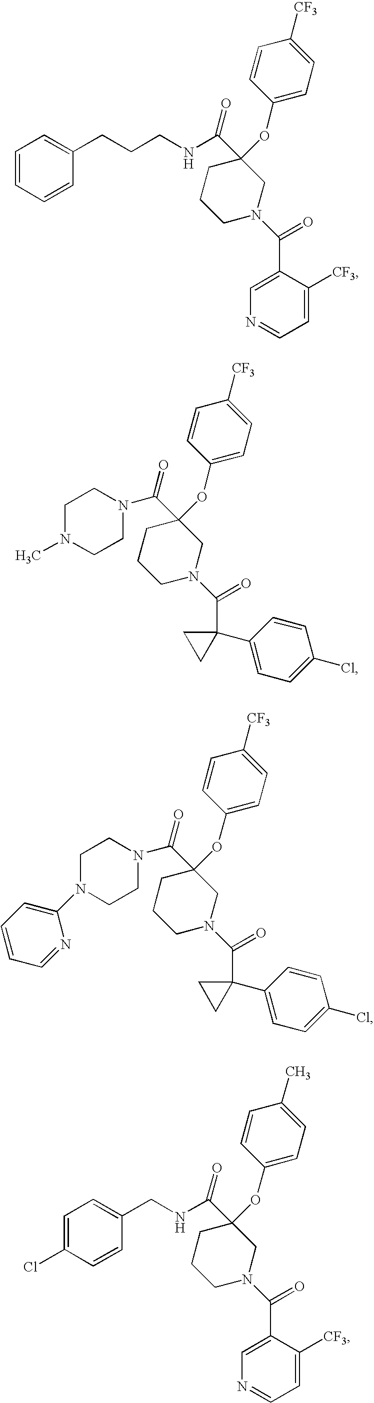

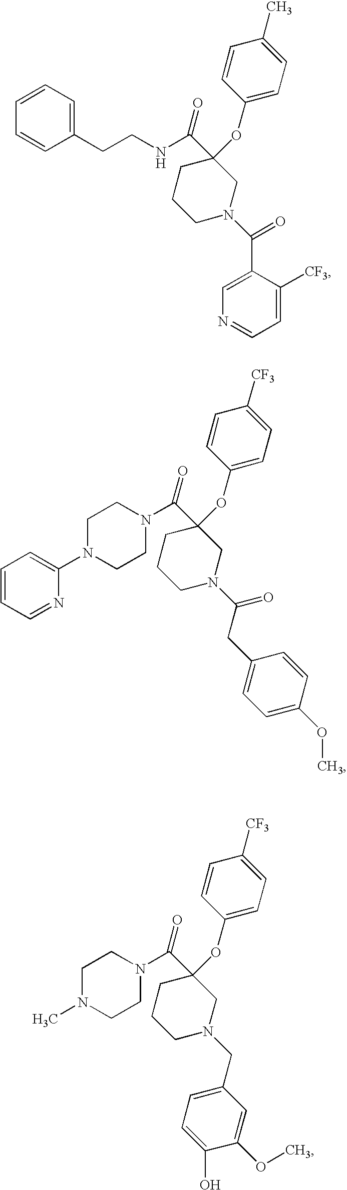

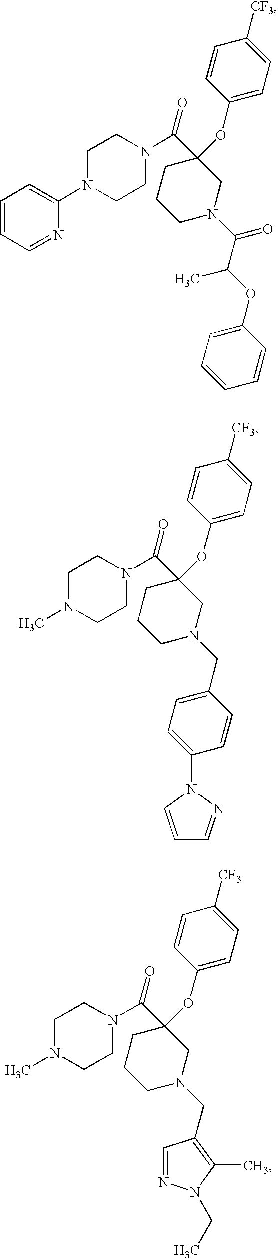

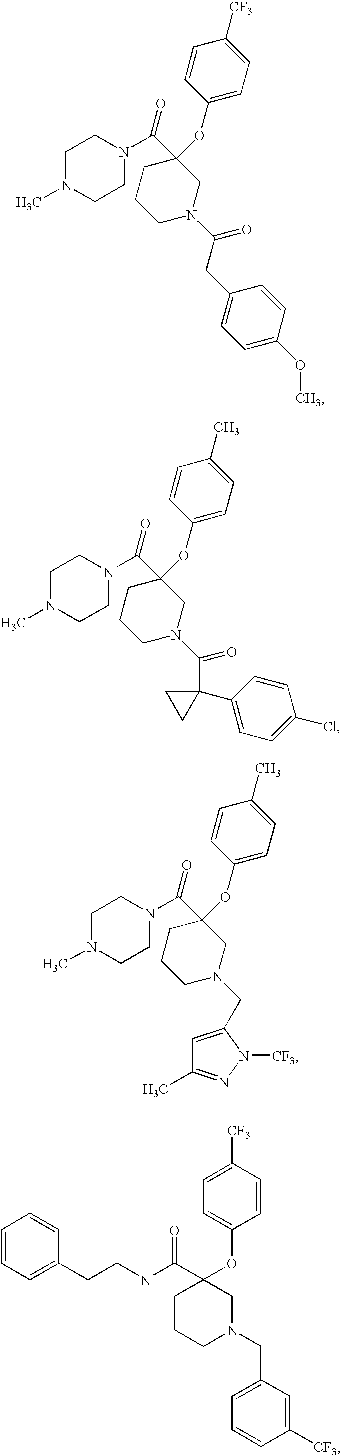

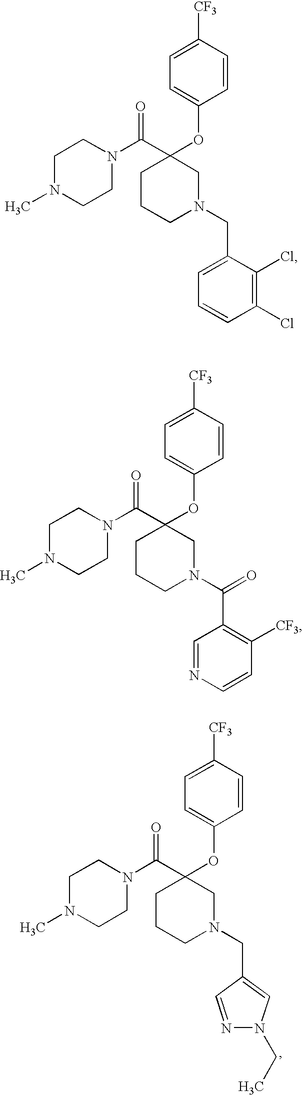

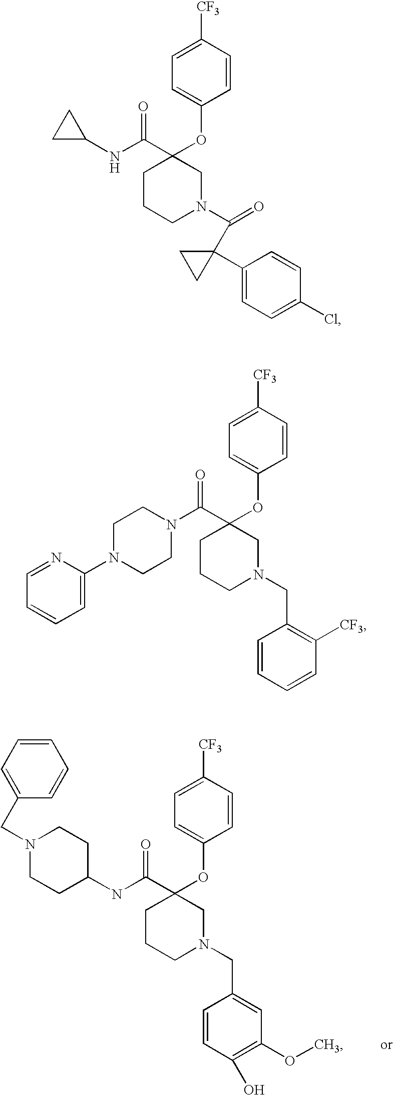

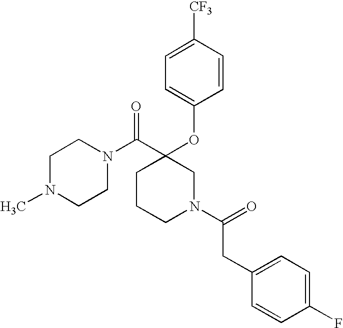

or a pharmaceutically acceptable salt, solvate, ester, or prodrug thereof to a patient in need of such inhibition.

### DETAILED DESCRIPTION OF THE INVENTION

In an embodiment, the present invention provides a method of inhibiting HDM2 protein comprising administering a therapeutically acceptable amount of at least one compound of the chemical structure illustrated above or a pharmaceutically acceptable salt, solvate, ester, or prodrug thereof to a patient in need of such inhibition.

In another embodiment, this invention discloses a method of treatment of one or more diseases associated with HDM2, comprising administering a therapeutically effective amount of at least one compound illustrated above to a patient in need of such treatment.

In yet another embodiment, the present invention provides a method of treatment of one or more diseases associated with P53, comprising administering a therapeutically effective amount of at least one compound illustrated above to a patient in need of such treatment.

In still another embodiment, this invention discloses a method of treatment of one or more diseases associated with HDM2 protein interacting with P53 protein, comprising administering a therapeutically effective amount of at least one compound illustrated above to a patient in need of such treatment.

In another embodiment, the present invention provides a method of treating one or more diseases associated with HDM2, comprising administering to a mammal in need of such treatment

an amount of a first compound, wherein said first compound is selected from the group of compounds illustrated above; and

an amount of at least one second compound, wherein said second compound is an anti-cancer agent different from the first compound;

wherein the amounts of the first compound and the second compound result in a therapeutic effect.

In yet another embodiment, this invention discloses a method of treating one or more diseases associated with P53 protein, comprising administering to a mammal in need of such treatment

an amount of a first compound, wherein said first compound is selected from the group of compounds illustrated above; and

an amount of at least one second compound, wherein said second compound being an anti-cancer agent different from the first compound;

wherein the amounts of the first compound and the second compound result in a therapeutic effect.

In still yet another embodiment, the present invention provides a method of treating one or more diseases associated with HDM2 protein interacting with P53 protein, comprising administering to a mammal in need of such treatment

an amount of a first compound, wherein said first compound is selected from the group of compounds illustrated above; and

an amount of at least one second compound, wherein said second compound being an anti-cancer agent different from the first compound;

wherein the amounts of the first compound and the second compound result in a therapeutic effect.

In another embodiment, this invention discloses a method of treating a disease selected from the group consisting of:

carcinoma, including, but not limited to, of the bladder, breast, colon, rectum, endometrium, kidney, liver, lung, head and neck, esophagus, gall bladder, cervix, pancreas, prostrate, larynx, ovaries, stomach, uterus, sarcoma and thyroid cancer;

hematopoietic tumors of the lymphoid lineage, including leukemia, acute lymphocytic leukemia, chronic Iymphocytic leukemia, acute lymphoblastic leukemia, B-cell lymphoma, T-cell lymphoma, Hodgkins lymphoma, non-Hodgkins lymphoma, hairy cell lymphoma, mantle cell lymphoma, myeloma, and Burkett's lymphoma;

hematopoetic tumors of myeloid lineage, including acute and chronic myelogenous leukemias, myelodysplastic syndrome and promyelocytic leukemia;

tumors of mesenchymal origin, including fibrosarcoma and rhabdomyosarcoma;

tumors of the central and peripheral nervous system, including astrocytoma, neuroblastoma, glioma, and schwannomas; and

other tumors, including melanoma, skin (non-melanomal) cancer, mesothelioma (cells), seminoma, teratocarcinoma, osteosarcoma, xenoderoma pigmentosum, keratoctanthoma, thyroid follicular cancer and Kaposi's sarcoma.

In yet another embodiment the method according this invention further comprising radiation therapy, surgery, chemotherapy, biological therapy, hormone therapy, photodynamic therapy, or bone marrow transplant.

### In still another embodiment, the present invention provides a method of treatment

wherein the anti-cancer agent described above, is selected from the group consisting of a cytostatic agent, cytotoxic agents, targeted therapeutic agents (small molecules, biologics, siRNA and microRNA) against cancer and neoplastic diseases,
anti-metabolites (such as methoxtrexate, 5-fluorouracil, gemcitabine, fludarabine, capecitabine);
alkylating agents, such as temozolomide, cyclophosphamide,
DNA interactive and DNA damaging agents, such as cisplatin, oxaliplatin, doxorubicin,
Ionizing irradiation, such as radiation therapy,
topoisomerase II inhibitors, such as etoposide, doxorubicin,
topoisomerase I inhibitors, such as irinotecan, topotecan,
tubulin interacting agents, such as paclitaxel, docetaxel, Abraxane, epothilones,
kinesin spindle protein inhibitors,
spindle checkpoint inhibitors,
Poly(ADP-ribose) polymerase (PARP) inhibitors
Matrix metalloprotease (MMP) inhibitors
Protease inhibitors, such as cathepsin D and cathepsin K inhibitors
Proteosome or ubiquitination inhibitors, such as bortezomib,
Activator of mutant P53 to restore its wild-type P53 activity
Adenoviral-P53
Bcl-2 inhibitors, such as ABT-263
Heat shock protein (HSP) modulators, such as geldanamycin and 17-AAG
Histone deacetylase (HDAC) inhibitors, such as vorinostat (SAHA),
sex hormone modulating agents,
   anti-estrogens, such as tamoxifen, fulvestrant,
   selective estrogen receptor modulators (SERM), such as raloxifene,
   anti-androgens, such as bicalutamide, flutamide
   LHRH agonists, such as leuprolide,
   5α-reductase inhibitors, such as finasteride,
   Cytochrome P450 C17 lysase (CYP450c17) inhibitors, such as Abiraterone
   aromatase inhibitors, such as letrozole, anastrozole, exemestane,
EGFR kinase inhibitors, such as geftinib, erlotinib, laptinib
dual erbB1 and erbB2 inhibitors, such as Lapatinib
multi-targeted kinases (serine/threonine and/or tyrosine kinase) inhibitors,
   ABL kinase inhibitors, imatinib and nilotinib, dasatinib
   VEGFR-1, VEGFR-2, PDGFR, KDR, FLT, c-Kit, Tie2, Raf, MEK and ERK
   inhibitors, such as sunitinib, soratenib, Vandetanib, pazopanib, Axitinib,
   PTK787,
   Polo-like kinase inhibitors,
   Aurora kinase inhibitors,
   JAK inhibitor
   c-MET kinase inhibitors
   Cyclin-dependent kinase inhibitors, such as CDK1 and CDK2 inhibitor SCH 727965

PI3K inhibitors

mTOR inhibitors, such as Rapamycin, Temsirolimus, and RAD001 and other anti-cancer (also know as anti-neoplastic) agents include but are not limited to ara-C, adriamycin, cytoxan, Carboplatin, Uracil mustard, Clormethine, Ifosfsmide, Melphalan, Chlorambucil, Pipobroman, Triethylenemelamine, Triethylenethiophosphoramine, Busulfan, Carmustine, Lomustine, Streptozocin, Dacarbazine, Floxuridine, Cytarabine, 6-Mercaptopurine, 6-Thioguanine, Fludarabine phosphate, Pentostatine, Vinblastine, Vincristine, Vindesine, Vinorelbine, Navelbine, Bleomycin, Dactinomycin, Daunorubicin, Doxorubicin, Epirubicin, teniposide, cytarabine, pemetrexed, Idarubicin, Mithramycin, Deoxycoformycin, Mitomycin-C, L-Asparaginase, Teniposide 17α-Ethinylestradiol, Diethylstilbestrol, Testosterone, Prednisone, Fluoxymesterone, Dromostanolone propionate, Testolactone, Megestrolacetate, Methylprednisolone, Methyltestosterone, Prednisolone, Triamcinolone, Chlorotrianisene, Hydroxyprogesterone, Aminoglutethimide, Estramustine, Flutamide Medroxyprogesteroneacetate, Toremifene, goserelin, Carboplatin, Hydroxyurea, Amsacrine, Procarbazine, Mitotane, Mitoxantrone, Levamisole, Drolloxafine, Hexamethylmelamine, Bexxar, Zevalin, Trisenox, Profimer, Thiotepa, Altretamine, Doxil, Ontak, Depocyt, Aranesp, Neupogen, Neulasta, Kepivance.
Farnesyl protein transterase inhibitors, such as, SARASAR™ (4-[2-[4-[(11R)-3,10-dibromo-8-chloro-6,11 -dihydro-5H-benzo[5,6]cyclohepta[1,2-b]pyridin-11-yl-]-1-piperidinyl]-2-oxoethyl]-piperidinecarboxamide, tipifarnib
interferons, such as Intron A, Peg-Intron,
anti-erbB1 antibodies, such as cetuximab, panitumumab,
anti-erbB2 antibodies, such as trastuzumab,
anti-CD52 antibodies, such as Alemtuzumab,
anti-CD20 antibodies, such as Rituximab
anti-CD33 antibodies, such as Gemtuzumab ozogamicin
anti-VEGF antibodies, such as Avastin,
TRIAL ligands, such as Lexatumumab, mapatumumab, and AMG-655 antibodies against CTLA-4, CTA1, CEA, CD5, CD19, CD22, CD30, CD44, CD44V6, CD55, CD56, EpCAM, FAP, MHCII, HGF, IL-6, MUC1, PSMA, TAL6, TAG-72, TRAILR, VEGFR, IGF-2, FGF,
anti-IGF-1R antibodies, such as SCH 717454.

Equivalent names that all represent Human Double Minute 2 protein described above include, but are not limited to HDM2, hDM2, hdm2, Hdm2, Human Double Minute 2, HDM-2, hDM-2, hdm-2, Hdm-2, Human Double Minute-2, hDM two, hdm two, Hdm two, Human Double Minute two, human double minute two, HDM-two, hDM-two, hdm-two, Hdm-two, Human Double Minute-two, human double minute-two, hDM Two, hdm Two, Hdm Two, Human Double Minute Two, human double minute Two, HDM-Two, hDM-Two, hdm-Two, Hdm-Two, Human Double Minute-Two or human double minute Two.

Likewise, Mouse Double Minute 2 protein can be represented the same way as the Human Double Minute Two protein described above, but replacing the “H” or “Human” with “M” or “Mouse” respectively.

Equivalent names that all represent P53 protein described above include, but are not limited to P-53, P53, p-53, P 53, p 53 or P53.

As used above, and throughout this disclosure, the following terms, unless otherwise indicated, shall be understood to have the following meanings:

“Patient” includes both human and animals.

“Mammal” means humans and other mammalian animals.

The term “purified”, “in purified form” or “in isolated and purified form” for a compound refers to the physical state of said compound after being isolated from a synthetic process (e.g. from a reaction mixture), or natural source or combination thereof. Thus, the term “purified”, “in purified form” or “in isolated and purified form” for a compound refers to the physical state of said compound after being obtained from a purification process or processes described herein or well known to the skilled artisan (e.g., chromatography, recrystallization and the like), in sufficient purity to be characterizable by standard analytical techniques described herein or well known to the skilled artisan.

It should also be noted that any carbon as well as heteroatom with unsatisfied valences in the text, schemes, examples and Tables herein is assumed to have the sufficient number of hydrogen atom(s) to satisfy the valences.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g, a drug precursor) that is transformed in vivo to yield a compound illustrated above or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a compound illustrated above or a pharmaceutically acceptable salt, hydrate or solvate of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N-(C₁-C₂)alkylamino(C₂-C₃)alkyl (such as β-dimethylaminoethyl), carbamoyl-(C₁-C₂)alkyl, N,N-di (C₁-C₂)alkylcarbamoyl-(C1-C2)alkyl and piperidino-, pyrrolidino- or morpholino(C₂-C₃)alkyl, and the like.

Similarly, if a compound illustrated above contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N-(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound illustrated above incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇) cycloalkyl, benzyl, or R-carbonyl is a natural α-aminoacyl or natural α-aminoacyl, —C(OH)C(O)OY¹ wherein Y¹ is H, (C₁-C₆)alkyl or benzyl, —C(OY²)Y³ wherein Y² is (C₁-C₄)alkyl and Y³ is (C₁-C₆)alkyl, carboxy(C₁-C₆)alkyl, amino(C₁-C₄)alkyl or mono-N— or di-N,N—(C₁-C₆)alkylaminoalkyl, —C(Y⁴)Y⁵ wherein Y⁴ is H or methyl and Y⁵ is mono-N— or di-N,N—(C₁-C₆)alkylamino morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

“Effective amount” or “therapeutically effective amount” is meant to describe an amount of compound or a composition of the present invention effective in inhibiting the above-noted diseases and thus producing the desired therapeutic, ameliorative, inhibitory, modulated, antagonistic, or preventative effect.

The compounds illustrated above can form salts which are also within the scope of this invention. Reference to a compound illustrated above herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound illustrated above contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful Salts of the compounds illustrated above may be formed, for example, by reacting a compound illustrated above with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et at Journal of Pharmaceutical Sciences (1977) 66(1) 1-19; P. Gould, International J. of Pharmaceutics (1986) 33 201-217; Anderson et al, The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g. methyl, ethyl, and butyl chlorides, bromides and iodides), dialkyl sulfates (e.g. dimethyl, diethyl, and dibutyl sulfates), long chain halides (e.g. decyl, lauryl, and stearyl chlorides, bromides and iodides), aralkyl halides (e.g. benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di(C₆₋₂₄)acyl glycerol.

Compounds illustrated above and salts, solvates, esters and prodrugs thereof, may exist in their tautomeric form (for example, as an amide or imino ether). All such tautomeric forms are contemplated herein as part of the present invention.

The compounds illustrated above may contain asymmetric or chiral centers, and, therefore, exist in different stereoisomeric forms. It is intended that all stereoisomeric forms of the compounds illustrated above as well as mixtures thereof, including racemic mixtures, form part of the present invention In addition, the present invention embraces all geometric and positional isomers. For example, if a compound illustrated above incorporates a double bond or a fused ring, both the cis- and trans-forms, as well as mixtures, are embraced within the scope of the invention.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds illustrated above may be atropisomers (e.g., substituted biaryls) and are considered as part of this invention. Enantiomers can also be separated by use of chiral HPLC column.

It is also possible that the compounds illustrated above may exist in different tautomeric forms, and all such forms are embraced within the scope of the invention. Also, for example, all keto-enol and imine-enamine forms of the compounds are included in the invention.

All stereoisomers (for example, geometric isomers, optical isomers and the like) of the present compounds (including those of the salts, solvates, esters and prodrugs of the compounds as well as the salts, solvates and esters of the prodrugs), such as those which may exist due to asymmetric carbons on various substituents, including enantiomeric forms (which may exist even in the absence of asymmetric carbons), rotameric forms, atropisomers, and diastereomeric forms, are contemplated within the scope of this invention, as are positional isomers (such as, for example, 4-pyridyl and 3-pyridyl). (For example, if a compound illustrated above incorporates a double bond or a fused ring, both the cis- and trans-forms, as well as mixtures, are embraced within the scope of the invention. Also, for example, all keto-enol and imine-enamine forms of the compounds are included in the invention,) Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to equally apply to the salt, solvate, ester and prodrug of enantiomers, stereoisomers, rotamers, tautomers, positional isomers, racemates or prodrugs of the inventive compounds.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

Certain isotopically-labelled compounds illustrated above (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labelled compounds illustrated above can generally be prepared by following procedures analogous to those disclosed in the Schemes and/or in the Examples hereinbelow, by substituting an appropriate isotopically labelled reagent for a non-isotopically labelled reagent.

Polymorphic forms of the compounds illustrated above, and of the salts, solvates, esters and prodrugs of the compounds illustrated above, are intended to be included in the present invention.

The compounds illustrated above can be inhibitors or antagonists of the Human Double Minute 2 protein or Mouse Double Minute 2 protein interaction with P-53 protein and it can be activators of the P-53 protein in cells. Furthermore, the pharmacological properties of the compounds illustrated above can be used to treat or prevent cancer, treat or prevent other disease states associated with abnormal cell proliferation, and treat or prevent diseases resulting from inadequate levels of P53 protein in cells.

Those skilled in the art will realize that the term “cancer” to be the name for diseases in which the body's cells may become abnormal and divide without control.

The compounds illustrated above can be useful to the treatment of a variety of cancers, including, but not limited to: carcinoma, including, but not limited to, of the bladder, breast, colon, rectum, endometrium, kidney, liver, lung, head and neck, esophagus, gall bladder, cervix, pancreas, prostrate, larynx, ovaries, stomach, uterus, sarcoma and thyroid cancer;

hematopoietic tumors of the lymphoid lineage, including leukemia, acute lymphocytic leukemia, chronic lymphocytic leukemia, acute lymphoblastic leukemia, B-cell lymphoma, T-cell lymphoma, Hodgkins lymphoma, non-Hodgkins lymphoma, hairy cell lymphoma, mantle cell lymphoma, myeloma, and Burkett's lymphoma;

hematopoetic tumors of myeloid lineage, including acute and chronic myelogenous leukemias, myelodysplastic syndrome and promyelocytic leukemia;

tumors of mesenchymal origin, including fibrosarcoma and rhabdomyosarcoma;

tumors of the central and peripheral nervous system, including astrocytoma, neuroblastoma, glioma, and schwannomas; and

other tumors, including melanoma, skin (non-melanomal) cancer, mesothelioma (cells), seminoma, teratocarcinoma, osteosarcoma, xenoderoma pigmentosum, keratoctanthoma, thyroid follicular cancer and Kaposi's sarcoma.

Due to the key role of P53 in the regulation of cellular apoptosis (cell death), the compounds of Formula (I) could act as agent to induce cell death which may be useful in the treatment of any disease process which features abnormal cellular proliferation eg, cancers of various origin and tissue types, inflammation, immunological disorders.

Due to the key role of HDM2 and P53 in the regulation of cellular proliferation, the compounds illustrated above could act as reversible cytostatic agents, which may be useful in the treatment of any disease process which features abnormal cellular proliferation, e.g., benign prostrate hyperplasia, familial adenomatosis polyposis, neuro-fibromatosis, atherosclerosis, pulmonary fibrosis, arthritis, psoriasis, glomerulonephritis, restenosis following angioplasty, or vascular surgery, hypertrophic scar formation, inflammatory bowel disease, transplantation rejection, endotoxic shock, and fungal infections.

Compounds illustrated above may also be useful in the chemoprevention of cancer. Chemoprevention is defined as inhibiting the development of invasive cancer by either blocking the initiating mutagenic event or by blocking the progression of pre-malignant cells that have already suffered an insult or inhibiting tumor relapse,

Compounds illustrated above may also be useful in inhibiting tumor angiogenesis and metastasis.

A preferred dosage is about 0.001 to 500 mg/kg of body weight/day of the compound illustrated above. An especially preferred dosage is about 0.01 to 25 mg/kg of body weight/day of a compound illustrated above, or a pharmaceutically acceptable salt, solvate, ester or prodrug of said compound.

If formulated as a fixed dose such combination products employ the compounds of this invention within the dosage range described herein and the other pharmaceutically active agent or treatment within its dosage range.

Compounds illustrated above may also be administered sequentially with known anticancer or cytotoxic agents when a combination formulation is inappropriate. The invention is not limited in the sequence of administration; compounds illustrated above may be administered either prior to or after administration of the known anticancer or cytotoxic agent. Such techniques are within the skills of the persons skilled in the art as well as attending physicians.

Preferred compounds can exhibit IC₅₀ or EC₅₀ values of less than about 15 μm, preferably about 0.001 μm to about 15.0 μm, more preferably about 0.001 μm to about 9 μm, still more preferably about 0.001 μm to about 3 μm.

In yet another embodiment, the present invention discloses methods for preparing pharmaceutical compositions comprising the compounds illustrated above as an active ingredient. In the pharmaceutical compositions and methods of the present invention, the active ingredients will typically be administered in admixture with suitable carrier materials suitably selected with respect to the intended form of administration, i.e. oral tablets, capsules (either solid-filled, semi-solid filled or liquid filled), powders for constitution, oral gels, elixirs, dispersible granules, syrups, suspensions, and the like, and consistent with conventional pharmaceutical practices. For example, for oral administration in the form of tablets or capsules, the active drug component may be combined with any oral non-toxic pharmaceutically acceptable inert carrier, such as lactose, starch, sucrose, cellulose, magnesium stearate, dicalcium phosphate, calcium sulfate, talc, mannitol, ethyl alcohol (liquid forms) and the like. Moreover, when desired or needed, suitable binders, lubricants, disintegrating agents and coloring agents may also be incorporated in the mixture. Powders and tablets may be comprised of from about 5 to about 95 percent inventive composition. Suitable binders include starch, gelatin, natural sugars, corn sweeteners, natural and synthetic gums such as acacia, sodium alginate, carboxymethylcellulose, polyethylene glycol and waxes. Lubricants in these dosage forms include boric acid, sodium benzoate, sodium acetate, sodium chloride, and the like. Disintegrants include starch, methylcellulose, guar gum and the like. Sweetening and flavoring agents and preservatives may also be included where appropriate. Some of the terms noted above, namely disintegrants, diluents, lubricants, binders and the like, are discussed in more detail below.

Additionally, the compositions of the present invention may be formulated in sustained release form to provide the rate controlled release of any one or more of the components or active ingredients to optimize the therapeutic effects, i.e. anti-cell proliferation activity and the like. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

Liquid form preparations include solutions, suspensions and emulsions. For example, water or water-propylene glycol solutions may be included for parenteral injections or sweeteners and pacifiers may be added for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier such as inert compressed gas, e.g. nitrogen.

For preparing suppositories, a low melting wax such as a mixture of fatty acid glycerides such as cocoa butter is first melted, and the active ingredient is dispersed homogeneously therein by stirring or similar mixing. The molten homogeneous mixture is then poured into convenient sized molds, allowed to cool to solidify.

Also included are solid form preparations which are intended to be converted, shortly before use, to liquid form preparations for either oral or parenteral administration. Such liquid forms include solutions, suspensions and emulsions.

The compounds of the invention may also be deliverable transdermally. The transdermal compositions may take the form of creams, lotions, aerosols and/or emulsions and can be included in a transdermal patch of the matrix or reservoir type as are conventional in the art for this purpose.

Preferably the compound is administered orally.

Preferably, the pharmaceutical preparation is in a unit dosage form. In such form, the preparation is subdivided into suitably sized unit doses containing appropriate quantities of the active components, e.g., an effective amount to achieve the desired purpose.

The quantity of the inventive active composition in a unit dose of preparation may be generally varied or adjusted from about 1.0 milligram to about 1,000 milligrams, preferably from about 1.0 to about 500 milligrams, and typically from about 1 to about 250 milligrams, according to the particular application. The actual dosage employed may be varied depending upon the patient's age, sex, weight and severity of the condition being treated. Such techniques are well known to those skilled in the art.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

Generally, the human oral dosage form containing the active ingredients can be administered 1 or 2 times per day. The amount and frequency of the administration will be regulated according to the judgment of the attending clinician. A generally recommended daily dosage regimen for oral administration may range from about 1.0 milligram to about 1,000 milligrams per day, in single or divided doses.

In another embodiment, this invention provides the use of pharmaceutical compositions comprising the above-illustrated compounds as an active ingredient to treat cancer, abnormal cell proliferation, and other HDM2 or P53 associated diseases.

The pharmaceutical compositions generally additionally comprise a pharmaceutically acceptable carrier diluent, excipient or carrier (collectively referred to herein as carrier materials).

Yet another aspect of this invention is a method of preparing a kit comprising an amount of at least one compound illustrated above, or a pharmaceutically acceptable salt, solvate, ester, or prodrug of said compound and an amount of at least one anticancer therapy and/or anti-cancer agent listed above, wherein the amounts of the two or more ingredients result in desired therapeutic effect.

Still another aspect of this invention is the use of a kit comprising an amount of at least one compound illustrated above, or a pharmaceutically acceptable salt, solvate, ester, or prodrug of said compound and an amount of at least one anticancer therapy and/or anti-cancer agent listed above, wherein the amounts of the two or more ingredients result in desired therapeutic effect to treat a mammal in need thereof.

Capsule—refers to a special container or enclosure made of methyl cellulose, polyvinyl alcohols, or denatured gelatins or starch for holding or containing compositions comprising the active ingredients. Hard shell capsules are typically made of blends of relatively high gel strength bone and pork skin gelatins. The capsule itself may contain small amounts of dyes, opaquing agents, plasticizers and preservatives.

Tablet—refers to a compressed or molded solid dosage form containing the active ingredients with suitable diluents. The tablet can be prepared by compression of mixtures or granulations obtained by wet granulation, dry granulation or by compaction.

Oral gels—refer to the active ingredients dispersed or solubilized in a hydrophillic semi-solid matrix.

Powders for constitution refer to powder blends containing the active ingredients and suitable diluents which can be suspended in water or juices.

Diluent—refers to substances that usually make up the major portion of the composition or dosage form. Suitable diluents include sugars such as lactose, sucrose, mannitol and sorbitol; starches derived from wheat, corn, rice and potato; and celluloses such as microcrystalline cellulose. The amount of diluent in the composition can range from about 10 to about 90% by weight of the total composition, preferably from about 25 to about 75%, more preferably from about 30 to about 60% by weight, even more preferably from about 12 to about 60%.

Disintegrants—refers to materials added to the composition to help it break apart (disintegrate) and release the medicaments. Suitable disintegrants include starches; “cold water soluble” modified starches such as sodium carboxymethyl starch; natural and synthetic gums such as locust bean, karaya, guar, tragacanth and agar; cellulose derivatives such as methylcellulose and sodium carboxymethylcellulose; microcrystalline celluloses and cross-linked microcrystalline celluloses such as sodium croscarmellose; alginates such as alginic acid and sodium alginate; clays such as bentonites; and effervescent mixtures. The amount of disintegrant in the composition can range from about 2 to about 15% by weight of the composition, more preferably from about 4 to about 10% by weight.

Binders—refers to substances that bind or “glue” powders together and make them cohesive by forming granules, thus serving as the “adhesive” in the formulation Binders add cohesive strength already available in the diluent or bulking agent. Suitable binders include sugars such as sucrose; starches derived from wheat, corn rice and potato; natural gums such as acacia, gelatin and tragacanth; derivatives of seaweed such as alginic acid, sodium alginate and ammonium calcium alginate; cellulosic materials such as methylcellulose and sodium carboxymethylcellulose and hydroxypropylmethylcellulose; polyvinylpyrrolidone; and inorganics such as magnesium aluminum silicate. The amount of binder in the composition can range from about 2 to about 20% by weight of the composition, more preferably from about 3 to about 10% by weight, even more preferably from about 3 to about 6% by weight.

Lubricant—refers to a substance added to the dosage form to enable the tablet, granules, etc. after it has been compressed, to release from the mold or die by reducing friction or wear Suitable lubricants include metallic stearates such as magnesium stearate, calcium stearate or potassium stearate; stearic acid; high melting point waxes; and water soluble lubricants such as sodium chloride, sodium benzoate, sodium acetate, sodium oleate, polyethylene glycols and d,l-leucine. Lubricants are usually added at the very last step before compression, since they must be present on the surfaces of the granules and in between them and the parts of the tablet press. The amount of lubricant in the composition can range from about 0.2 to about 5% by weight of the composition, preferably from about 0.5 to about 2%, more preferably from about 0.3 to about 1.5% by weight.

Glidents—materials that prevent caking and improve the flow characteristics of granulations, so that flow is smooth and uniform. Suitable glidents include silicon dioxide and talc. The amount of glident in the composition can range from about 0.1% to about 5% by weight of the total composition, preferably from about 0.5 to about 2% by weight.

Coloring agents—excipients that provide coloration to the composition or the dosage form. Such excipients can include food grade dyes and food grade dyes adsorbed onto a suitable adsorbent such as clay or aluminum oxide. The amount of the coloring agent can vary from about 0.1 to about 5% by weight of the composition, preferably from about 0.1 to about 1%.

In yet another embodiment, the present invention discloses methods for preparing pharmaceutical compositions comprising the compounds illustrated above as an active ingredient. In the pharmaceutical compositions and methods of the present invention, the active ingredients will typically be administered in admixture with suitable carrier materials suitably selected with respect to the intended form of administration, i.e. oral tablets, capsules (either solid-filled, semi-solid filled or liquid filled), powders for constitution, oral gels, elixirs, dispersible granules, syrups, suspensions, and the like, and consistent with conventional pharmaceutical practices. For example, for oral administration in the form of tablets or capsules, the active drug component may be combined with any oral non-toxic pharmaceutically acceptable inert carrier, such as lactose, starch, sucrose, cellulose, magnesium stearate, dicalcium phosphate, calcium sulfate, talc, mannitol, ethyl alcohol (liquid forms) and the like. Moreover, when desired or needed, suitable binders, lubricants, disintegrating agents and coloring agents may also be incorporated in the mixture. Powders and tablets may be comprised of from about 5 to about 95 percent inventive composition. Suitable binders include starch, gelatin, natural sugars, corn sweeteners, natural and synthetic gums such as acacia, sodium alginate, carboxymethylcellulose, polyethylene glycol and waxes. Lubricants in these dosage forms include boric acid, sodium benzoate, sodium acetate, sodium chloride, and the like. Disintegrants include starch, methylcellulose, guar gum and the like. Sweetening and flavoring agents and preservatives may also be included where appropriate. Some of the terms noted above, namely disintegrants, diluents, lubricants, binders and the like, are discussed in more detail below.

Additionally, the compositions of the present invention may be formulated in sustained release form to provide the rate controlled release of any one or more of the components or active ingredients to optimize the therapeutic effects, i.e. anti-cell proliferation activity and the like. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

Liquid form preparations include solutions, suspensions and emulsions. For example, water or water-propylene glycol solutions may be included for parenteral injections or sweeteners and pacifiers may be added for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier such as inert compressed gas, e.g. nitrogen.

For preparing suppositories, a low melting wax such as a mixture of fatty acid glycerides such as cocoa butter is first melted, and the active ingredient is dispersed homogeneously therein by stirring or similar mixing. The molten homogeneous mixture is then poured into convenient sized molds, allowed to cool to solidify.

Also included are solid form preparations which are intended to be converted, shortly before use, to liquid form preparations for either oral or parenteral administration. Such liquid forms include solutions, suspensions and emulsions.

The compounds of the invention may also be deliverable transdermally. The transdermal compositions may take the form of creams, lotions, aerosols and/or emulsions and can be included in a transdermal patch of the matrix or reservoir type as are conventional in the art for this purpose.

Preferably the compound is administered orally.

Preferably, the pharmaceutical preparation is in a unit dosage form. In such form, the preparation is subdivided into suitably sized unit doses containing appropriate quantities of the active components, e.g., an effective amount to achieve the desired purpose.

The quantity of the inventive active composition in a unit dose of preparation may be generally varied or adjusted from about 1.0 milligram to about 1,000 milligrams, preferably from about 1.0 to about 500 milligrams, and typically from about 1 to about 250 milligrams, according to the particular application. The actual dosage employed may be varied depending upon the patient's age, sex, weight and severity of the condition being treated. Such techniques are well known to those skilled in the art.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

Generally, the human oral dosage form containing the active ingredients can be administered 1 or 2 times per day. The amount and frequency of the administration will be regulated according to the judgment of the attending clinician. A generally recommended daily dosage regimen for oral administration may range from about 1.0 milligram to about 1,000 milligrams per day, in single or divided doses.

Bioavailability—refers to the rate and extent to which the active drug ingredient or therapeutic moiety is absorbed into the systemic circulation from an administered dosage form as compared to a standard or control.

Conventional methods for preparing tablets are known. Such methods include dry methods such as direct compression and compression of granulation produced by compaction, or wet methods or other special procedures. Conventional methods for making other forms for administration such as, for example, capsules, suppositories and the like are also well known.

The invention disclosed herein is exemplified by the following preparations and examples which should not be construed to limit the scope of the disclosure. Alternative mechanistic pathways and analogous structures will be apparent to those skilled in the art.

### EXAMPLES

Unless otherwise stated, the following abbreviations have the stated meanings in the Examples below:
N,N-diisoproplyethylamine: iPr2NEt
High Resolution Mass Spectrometry: HRMS
High Performance Liquid Chromatography: HPLC
Low Resolution Mass Spectrometry: LRMS
Nanomolar: nM
Inhibitor constant for substrate/receptor complex: Ki
polystyrene-bound carbodiimide resin: PS-CDI
O-(Benzotriazol-1-yl)-N,N,N′,N′-tetramethyluronium tetrafluoroborate: TBTU
Proton Nuclear Magnetic Resonance: ¹H NMR
Liquid Chromatography Mass Spectrometry data are presented, analyses was performed using an Applied Biosystems API-100 mass spectrometer and Shimadzu
SCL-10A LC column: (observed parent ion (M+) is given.): LCMS:
Efficacious concentration that achieves 50% of maximal activity: EC₅₀
Inhibitory concentration that achieves 50% of maximal activity: IC₅₀
milliliters: mL
millimoles: mmol
microliters: μl
grams: g
milligrams: mg
room temperature: rt (ambient): about 25° C.

Compounds used in the present invention illustrated above are prepared by methods known in the art, for example, according to the general reaction sequence shown in Scheme 1 and the preparative example following it:

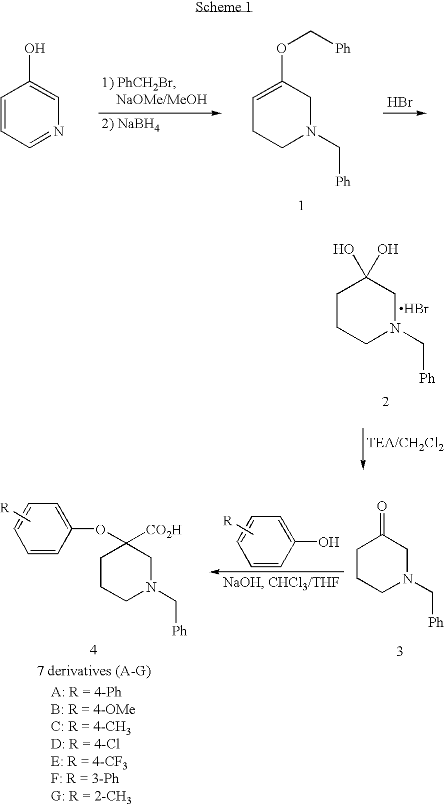

### Step 1:

### Benzyl-1,2,5,6-tetrahydro-3-pyridyl benzyl ether (1)

To a solution of sodium methoxide (62.4 g, 1.16 mol) prepared from 600 mL of methanol was added 3-hydroxypyridine (100 g, 1.05 mol). Upon addition of benzyl bromide (375 mL, 3.15 mol) the solution was refluxed for overnight. After cooling to room temperature, sodium borohydride (79.4 g, 2.1 mol) was added in portions. The solvent was removed in vacuo and the residue was stirred with water 650 mL, potassium carbonate 64 g, and ether 800 mL for 1 hour to give two homogeneous liquid phase. The ether phase was isolated, dried over potassium carbonate and evaporated in vacuo to give brown oil. To a solution of this oil in ether 20 mL was added slowly and with vigorous stirring pet. ether 2.1 L and celite 521 35 g, and stirring was continued for additional 30 min. The filtrate was evaporated in-vacuo to give Benzyl-1,2,5,6-tetrahydro-3-pyridyl benzyl ether as the desired material (294 g, 100%).

### Step 2:

### 1-Benzyl-3,3-dihydroxypiperidine hydrobromide (2)

A solution of Benzyl-1,2,5,6-tetrahydro-3-pyridyl benzyl ether (1, 294 g, 1.05 mol) in 48% HBr (385 mL, 7.77 mol) was refluxed for 3 hours. After cooling to room temperature the reaction mixture was extracted with ether (4×300 mL). The aqueous layer was evaporated in vacuo to give an oil, which was crystallized (butanone) to give 1-Benzyl-3,3-dihydroxypiperidine hydrobromide as the desired material (129 g. 43%).

### Step 3:

### 1-Benzyl-3-piperidone (3)

To a 1-benzyl-3-piperidone HBr salt (2, 464 g, 1.61 mol) suspended in CH₂Cl₂ 3.5 L was added triethylamine (247 mL, 1.77 mol), then stirred for 3 hours. The resultant mixture was washed with H₂O (3.5 L×2) and 4 L of brine, then dried over MgSO₄, filtered and CH₂Cl₂ was removed to give 1-Benzyl-3-piperidone as the desired material (305 g, 100%).

### Step 4:

Use of 7 phenols to prepare 7 derivatives (4):

### A. 1-Benzyl-3-(biphenyl-4-yloxy)-piperidine-3-carboxylic acid

Sodium hydroxide (212 g, 5.28 mol) was added to stirred solution of 4-phenyl phenol (100 g, 0.588 mol) in anhydrous tetrahydrofuran 3 L. After 3 hours, 1-benzyl-3-piperidone (3, 444 g, 2.35 mol) was added, the mixture was cooled to 0° C. and anhydrous chloroform (282 mL, 2.52 mol) was added dropwise. The reaction mixture was maintained at 0° C. for 1 hour and then heated to 40° C. for 2˜3 h, stirred overnight at room temperature. Tetrahydrofuran was removed under reduced pressure. The residue was suspended in water (3 L) and washed with diethyl ether (3 L). The aqueous layer was acidified with 6N HCl to pH 5, filtered and washed with CH₂Cl₂ to give 1-Benzyl-3-(biphenyl-4-yloxy)-piperidine-3-carboxylic acid as the desired material (156 g, 68.5%).

### B. 1-Benzyl-3-(4-methoxy-phenoxy)-piperidine-3-carboxylic acid

Sodium hydroxide (290 g, 7.26 mol) was added to stirred solution of 4-Methoxyphenol (100 g, 0.8 mol) in anhydrous tetrahydrofuran (3 L). After 3 hours, 1-benzyl-3-piperidone (3, 610 g, 3.22 mol) was added, the mixture was cooled to 0° C. and anhydrous chloroform (386 mL, 4.84 mol) was added dropwise. The reaction mixture was maintained at 0° C. for 1 hour and then heated to 40° C. for 2˜3 h, stirred overnight at room temperature. Tetrahydrofuran was removed under reduced pressure. The residue was suspended in water (3 L) and washed with diethyl ether (3 L). The aqueous layer was acidified with 6N HCl to pH 5, filtered and washed with CH₂Cl₂ to give a 1-Benzyl-3-(4-methoxy-phenoxy)-piperidine-3-carboxylic acid as the desired material (135 g, 49.0%)

### C. 1-Benzyl-3-p-tolyloxy-piperidine-3-carboxylic acid

Sodium hydroxide (260 g, 6.5 mol) was added to stirred solution of p-cresol (78 g, 0.72 mol) in anhydrous tetrahydrofuran 3 L. After 3 hours, 1-benzyl-3-piperidone (3, 547 g, 2.89 mol) was added, the mixture was cooled to 0° C. and anhydrous chloroform (347 mL, 4.33 mol) was added dropwise. The reaction mixture was maintained at 0° C. for 1 hour and then heated to 40° C. for 2˜3 h, stirred for overnight at room temperature. Tetrahydrofuran was removed under reduced pressure. The residue was suspended in water (2.5 L) and washed with diethyl ether (2.5 L). The aqueous layer was acidified with 6N HCl to pH 5, filtered and washed with CH₂Cl₂ to give 1-Benzyl-3-p-tolyloxy-piperidine-3-carboxylic acid as the desired material (120 g, 52.0%).

### D. 1-Benzyl-3-(4-chloro-phenoxy)-piperidine-3-carboxylic acid

Sodium hydroxide (381 g, 9.53 mol) was added to stirred solution of 4-Chlorophenol (136 g, 1.06 mol) in anhydrous tetrahydrofuran (3 L). After 3 hours, 1-benzyl-3-piperidone (3, 801 g, 4.23 mol) was added, the mixture was cooled to 0° C. and anhydrous chloroform (508 mL, 6.35 mol) was added dropwise. The reaction mixture was maintained at 0° C. for 1 hour and then heated to 40° C. for 2˜3 h, stirred overnight at room temperature. Tetrahydrofuran was removed under reduced pressure. The residue was suspended in water (3 L) and washed with diethyl ether (3 L). The aqueous layer was acidified with 6N HCl to pH 5, filtered and washed with CH₂Cl₂ to give 1-Benzyl-3-(4-chloro-phenoxy)-piperidine-3-carboxylic acid as the desired material (210 g, 57.4%).

### E. 1-Benzyl-3-(4-trifluoromethyl-phenoxy)-piperidine-3-carboxylic acid

Sodium hydroxide (222 g, 5.55 mol) was added to stirred solution of 4-hydroxybenzotri-fluoride (100 g, 0.62 mol) in anhydrous tetrahydrofuran (3 L). After 3 hours, 1-benzyl-3-piperidone (3, 467 g, 2.47 mol) was added, the mixture was cooled to 0° C. and anhydrous chloroform (296 mL, 3.7 mol) was added dropwise. The reaction mixture was maintained at 0° C. for 1 hour and then allowed to 40° C. for 2˜3 h, stirred for overnight at room temperature. Tetrahydrofuran was removed under reduced pressure. The residue was suspended in water (3 L) and washed with diethyl ether (3 L). The aqueous layer was acidified with 6 N HCl by pH 7, filtered and washed with CH₂Cl₂ to give 1-Benzyl-3-(4-trifluoromethyl-phenoxy)-piperidine-3-carboxylic acid as the desired material (146 g, 62.4%)

### F. 1-Benzyl-3-(biphenyl-3-yloxy)-piperidine-3-carboxylic acid

Sodium hydroxide (212 g, 5.28 mol) was added to stirred solution of 3-phenyl phenol (100 g, 0.588 mol) in anhydrous tetrahydrofuran (3 L). After 3 hours, 1-benzyl-3-piperidone (3, 444 g, 2.35 mol) was added, the mixture was cooled to 0° C. and anhydrous chloroform (282 mL, 2.52 mol) was added dropwise. The reaction mixture was maintained at 0° C. for 1 hour and then allowed to 40° C. for 2˜3 hours, stirred for overnight at room temperature. Tetrahydrofuran was removed under reduced pressure. The residue was suspended in water (3 L) and washed with diethyl ether (3 L). The aqueous layer was acidified with 6N HCl to pH 5, filtered and washed with CH₂Cl₂ to give a 1-Benzyl-3-(biphenyl-3-yloxy)-piperidine-3-carboxylic acid as the desired material (80 g, 35.2%).

### G. 1-Benzyl-3-o-tolyloxy-piperidine-3-carboxylic acid

Sodium hydroxide (332 g, 8.3 mol) was added to stirred solution of o-Cresol (100 g, 0.925 mol) in anhydrous tetrahydrofuran (2 L). After 3 hours, 1-benzyl-3-piperidone (3, 700 g, 3.67 mol) was added, the mixture was cooled to 0° C. and anhydrous chloroform (440 mL, 5.55 mol) was added dropwise. The reaction mixture was maintained at 0° C. for 1 hour and then heated to 60° C. for 2˜3 h, stirred overnight at room temperature. Tetrahydrofuran was removed under reduced pressure. The residue was suspended in water (2.5 L) and washed with diethyl ether (2.5 L). The aqueous layer was acidified with 6N HCl by pH 7, extracted with methylene chloride and dried over MgSO₄. The crude mixture (380 g) was suspended in ethyl acetate (4 L) and cyclohexylamine (170 mL) was added. The mixture was stirred for 1 hour and stored in refrigerator for 2 days. The precipitate was filtered and washed with CH₂Cl₂. The salt (100 g) was suspended in methylene chloride (1 L), 6N HCl (43 mL, 0.26 mol) was added, then solid was filtered and washed with methylene chloride and diethyl ether to give 1-Benzyl-3-o-tolyloxy-piperidine-3-carboxylic acid as the desired material (40 g, 13.3%)

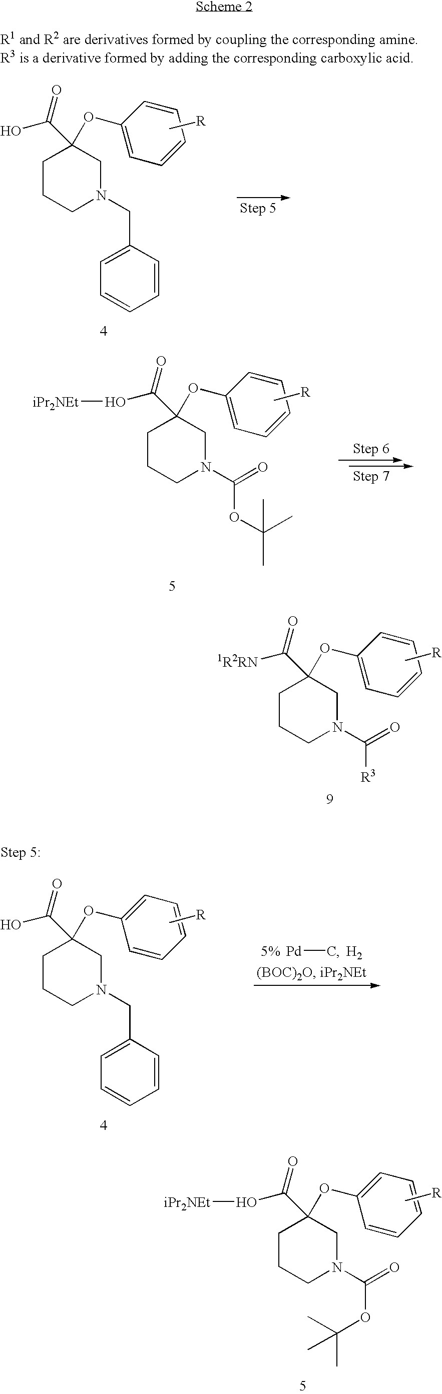

To 4 (1 eq, 18 mmol, 6.9 g) and N,N-diisopropylethylamine (5 eq, 91 mmol, 15.8 mL) completely dissolved in 25% ethanol/75% ethyl acetate (400 mL) was added a solution of di-tertbutyl dicarbonate (1 eq, 18 mmol, 4.0 g) in ethyl acetate (50 mL) followed by 5% palladium on carbon (30 wt %, 2.0 g) at room temperature. The reaction vessel was sealed with a septum, purged with argon, and hydrogen gas was bubbled through the solvent for 2 minutes. The reaction mixture was stirred under a hydrogen gas atmosphere at room temperature for 15 hours, then filtered through celite and concentrated in vacuo to give 5 as an off-white solid in the form of the corresponding diisopropylethylammonium salt which was used without further purification.

### Step 6:

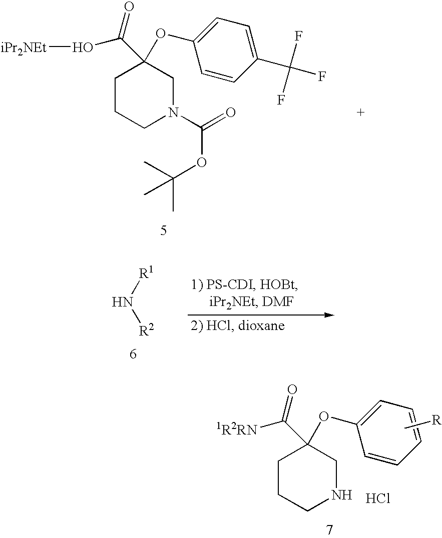

To 5, the product of step 1, (0.1 mmol) in N,N-dimethylformamide (0.67 mL) and N,N-diisopropylethylamine (3.0 eq, 0.3 mmol, 52 uL) was added 1-hydroxybenzotriazole (1.0 eq, 0.1 mmol, 14 mg), 6 (1.5 eq, 0.15 mmol, 29 mg), and polystyrene-bound carbodiimide resin, loading: 1.3 mmol/g (3.0 eq, 0.3 mmol, 231 mg). The mixture was shaken overnight at room temperature and scavenged with MP-trisamine and MP-isocyanate resins (excess) in tetrahydrofuran (3 mL) for 2 h. The resins were removed by filtration and the solvent removed in vacuo. The crude reaction mixture was dissolved in 4N hydrochloric acid in 1,4-dioxane (3 mL) and shaken at room temperature for 2 hours followed by evaporation in vacuo. The crude residue (7) was used without further purification.

### Step 7:

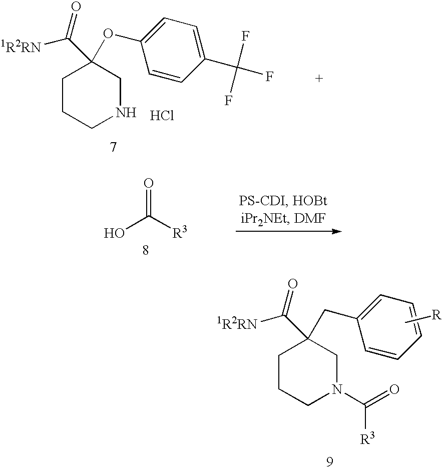

To 7, the product of step 2, (1.0 eq, 0.2 mmol, 100 mg), 8 (1.5 eq, 0.3 mmol, 58 mg), and 1-hydroxybenzotriazole (1.0 eq, 0.2 mmol, 27 mg) in N,N-dimethylformamide (6.7 mL) and N,N-diisopropylethylamine (4.0 eq, 0.8 mmol, 140 uL) was added. Polystyrene-bound carbodiimide resin, loading: 1.3 mmol/g (3.0 eq, 0.6 mmol, 462 mg) was added and shaken overnight at room temperature. The resin was removed by filtration, the solvent removed in vacuo, and the crude residue was purified by HPLC-MS to give the target compound of preparation 1 as the TFA-salt. The solid was dissolved in an acetonitrile/H₂O solution (1:1, 1.0 mL total) and 1.0 N hydrochloric acid (200 uL) and lyophilized to give the target compound of preparation 1 (9) in the form of the corresponding hydrochloric acid-salt (M+: 636.2) The inventive compounds can readily be evaluated to determine activity at the HDM2 protein by known methods such as the fluorescence polarization screening assay that measures the inhibitory concentration that achieves 50% of maximal activity (FP IC₅₀) and the dissociation constant for inhibitor binding (FP Ki). [Zhang et al., J. Analytical Biochemistry 331: 138-146 (2004)].

Additionally, compounds are tested for activity at the HDM2 protein using the Cell Viability Assay, which measures the number of viable cells in culture after treatment with the inventive compound for a certain period of time e.g. 72 hours based on quantitation of the ATP present (Cell Viability. IC₅₀). [CellTiter-Glo® Luminescent Cell Viability Assay from Promega].

Compounds of the present application exhibit FP IC₅₀, FP Ki, and Cell Viability IC₅₀ values less than 50.0 μM.

Compounds used in this invention were prepared by essentially the same procedures given in the preparative examples above.

The HDM2 inhibitory activities for representative compounds are shown in Table 1 below.

TABLE 1

| Compound | | |
|---|---|---|
| No. | Structure | FP IC50 (μM) |
| 1 | 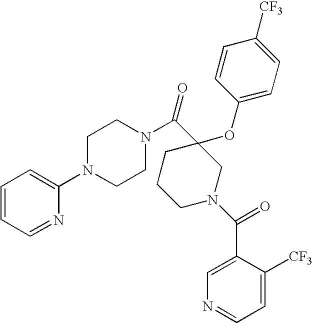 | 2.3 |
| 2 | 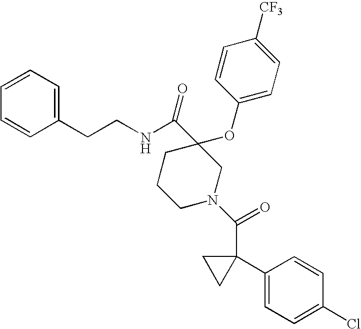 | 1.4 |
| 3 | 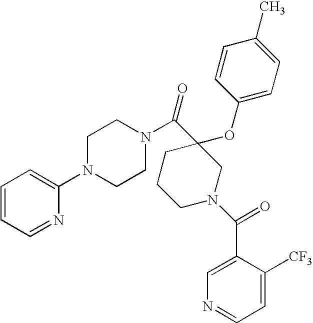 | 1.5 |

From these test results, it would be apparent to the skilled artisan that the compounds of the invention have utility in treating diseases associated with HDM2 protein and inadequate levels of P53 protein, which include, but is not limited to diseases that result in excessive cell proliferation such as cancer.

What is claimed is:

1. A method of inhibiting HDM2 protein comprising administering a therapeutically acceptable amount of at least one compound of the following chemical structure: 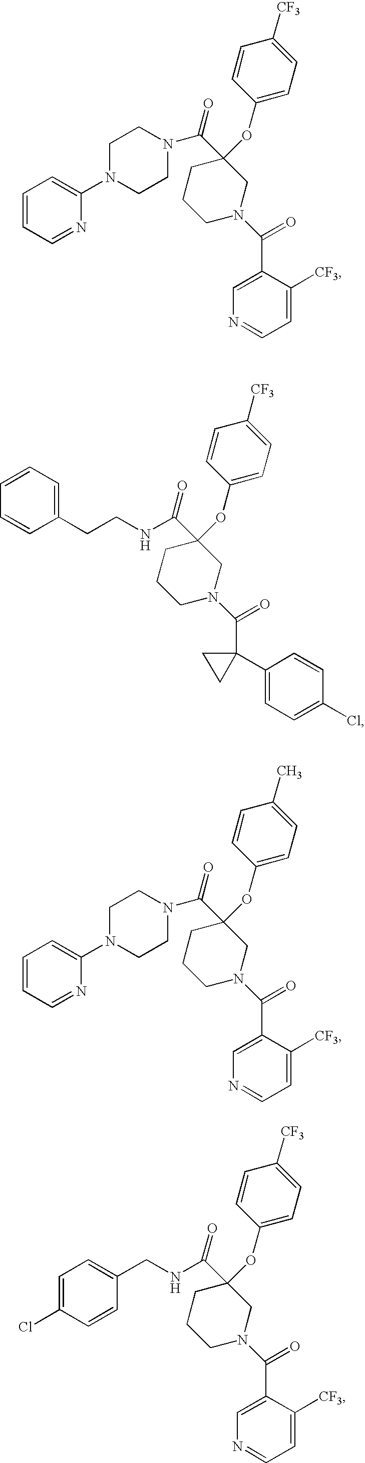 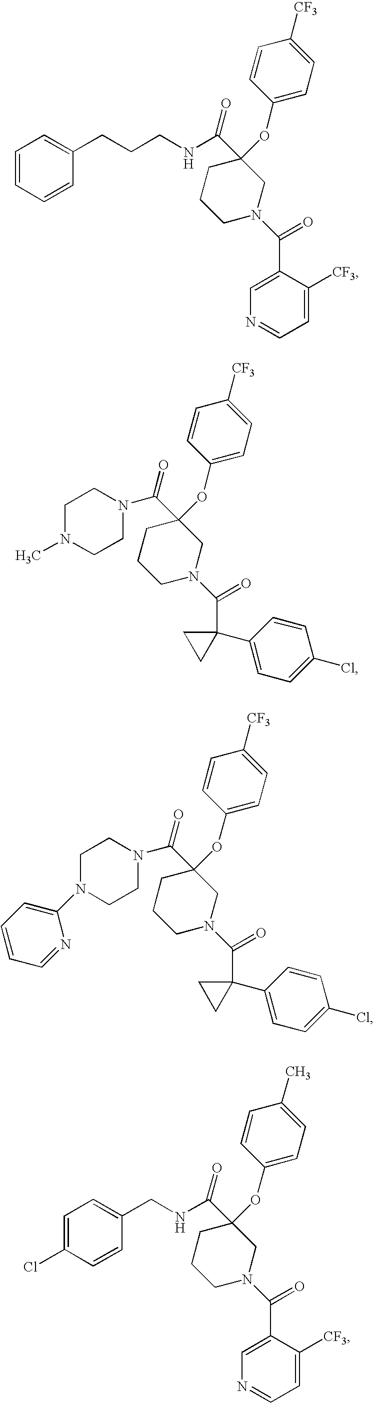 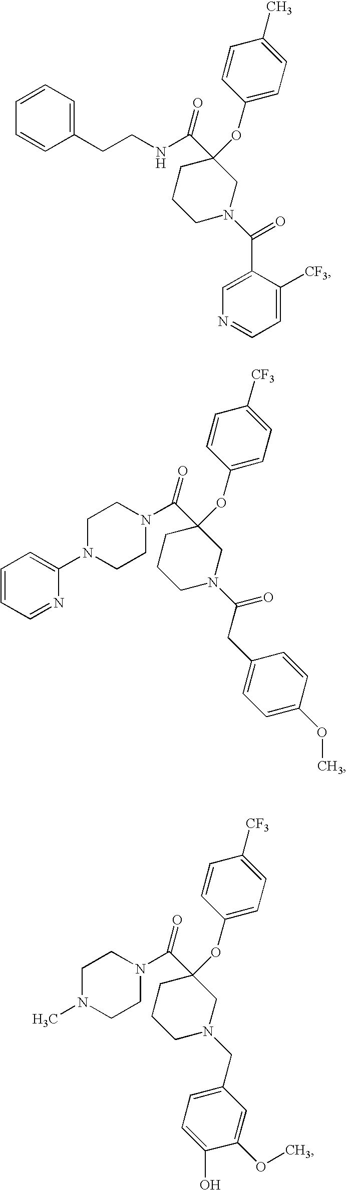 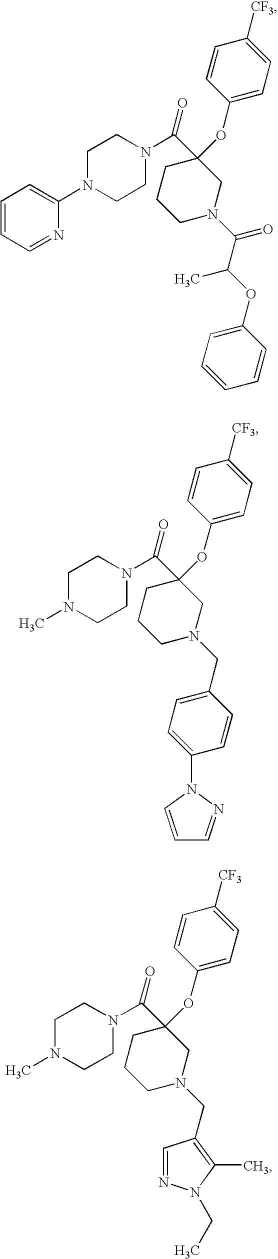 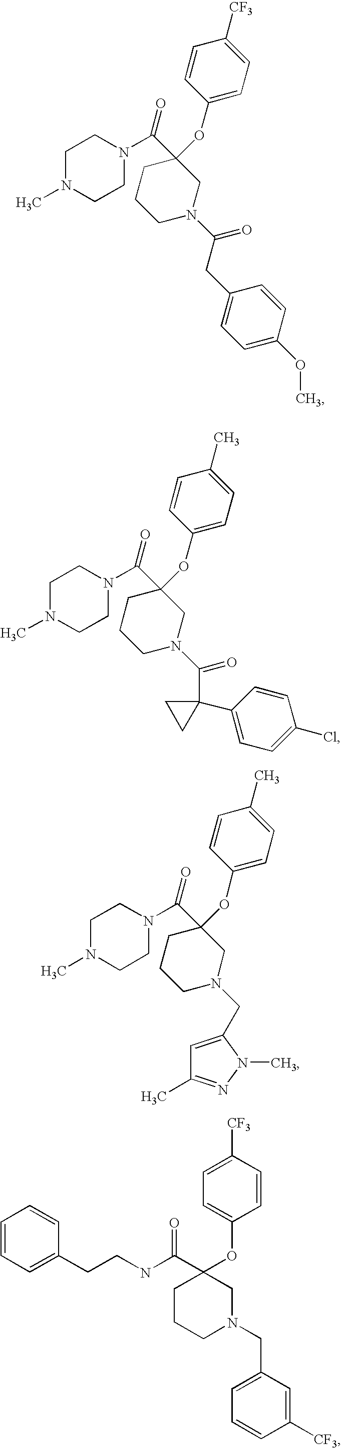 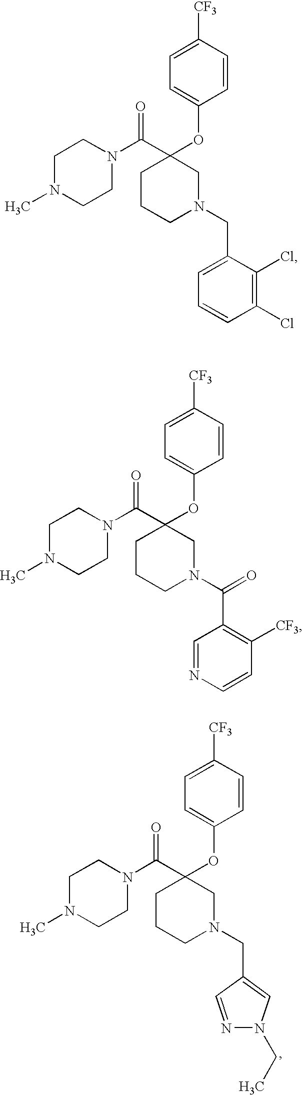 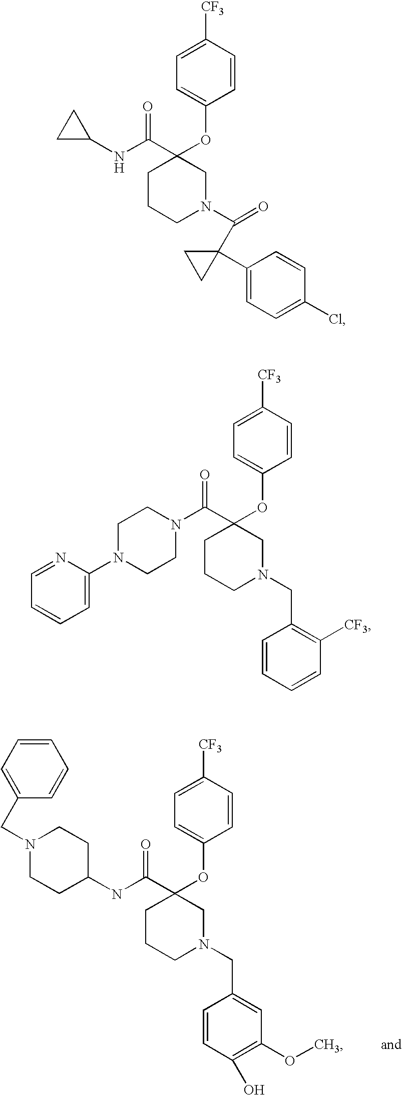 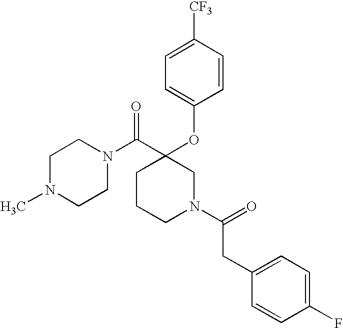
or a pharmaceutically acceptable salt, solvate, ester, or prodrug thereof to a mammal in need of such inhibition.

2. A method of treating or preventing one or more diseases associated with HDM2, comprising administering a therapeutically effective amount of at least one compound of the following structure: 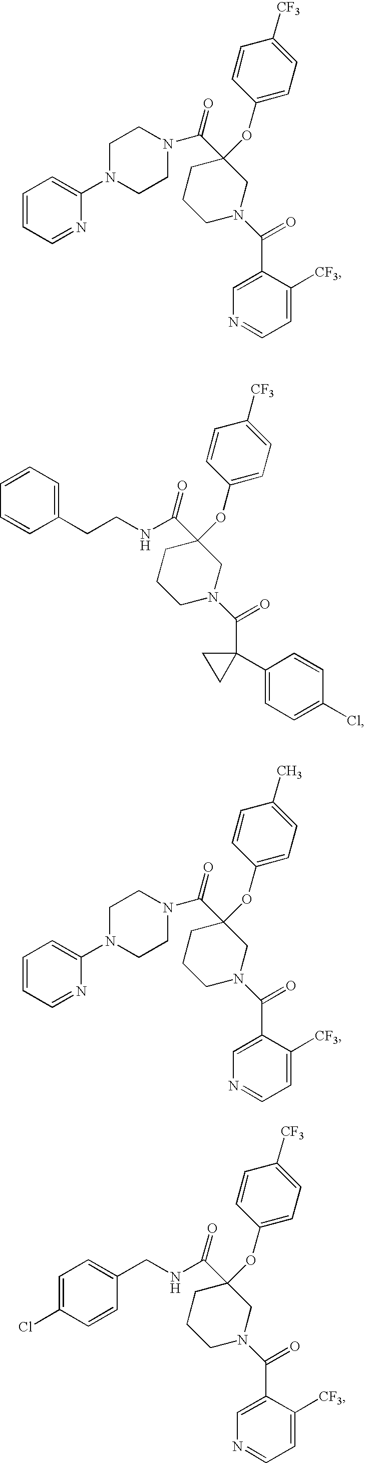 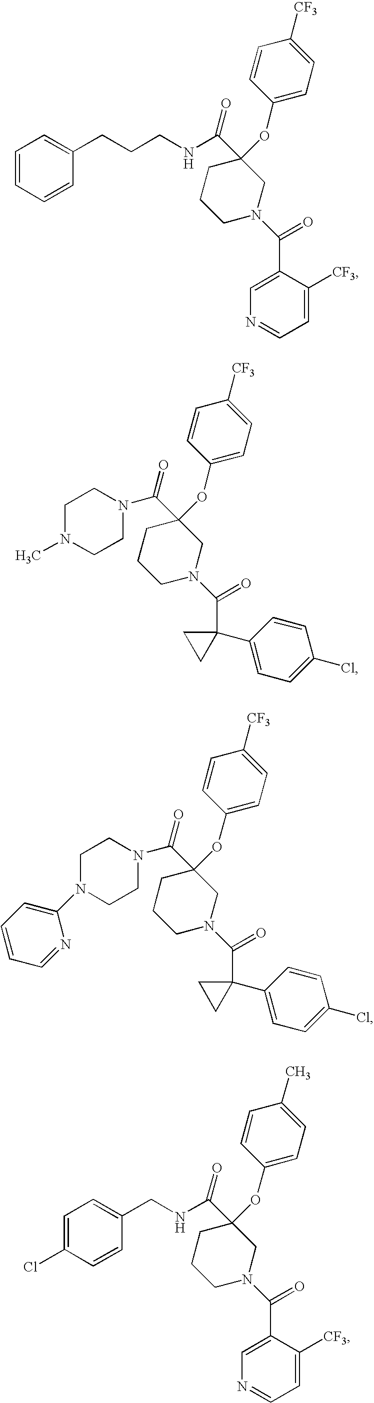 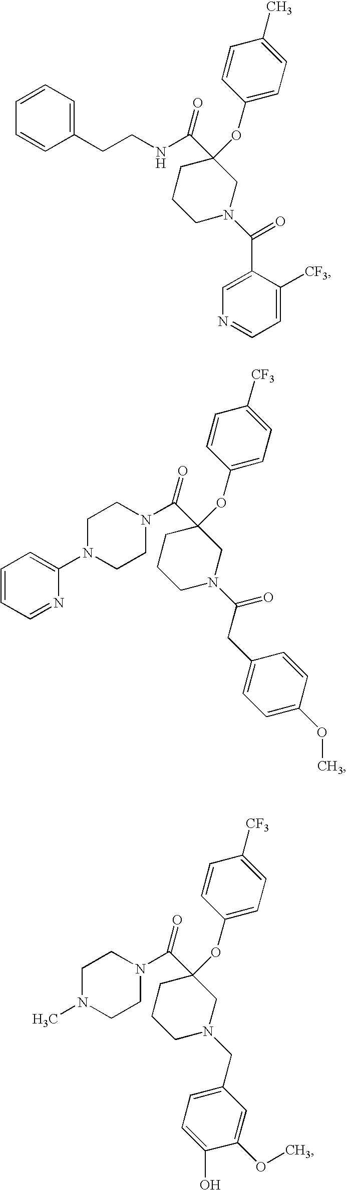 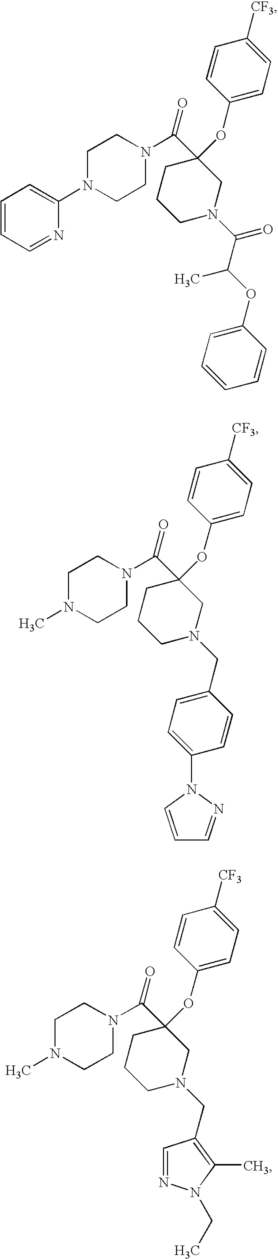 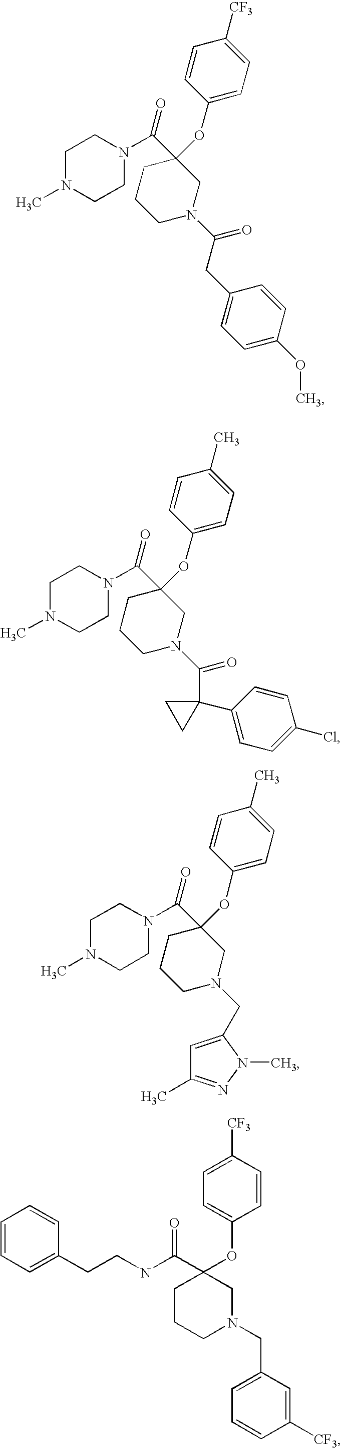 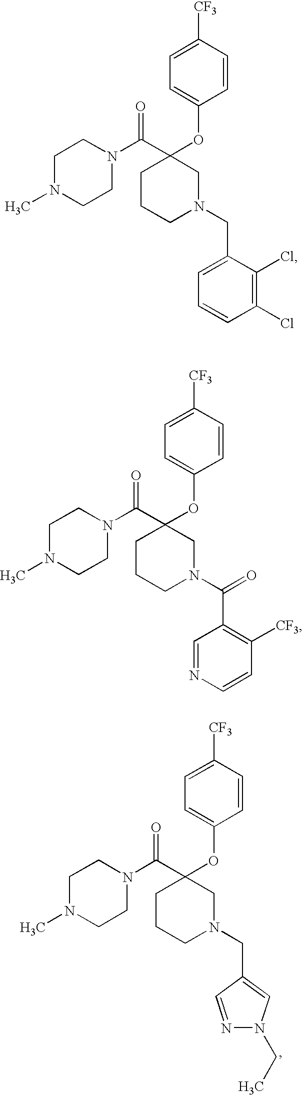 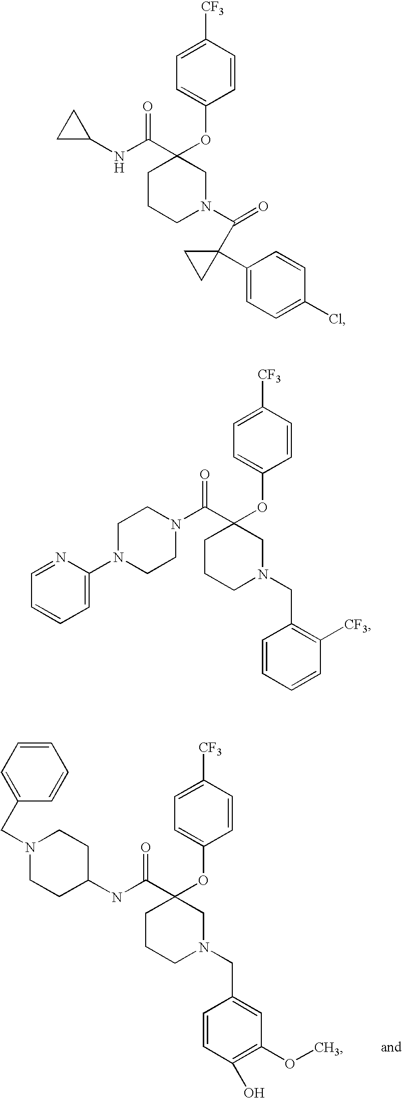 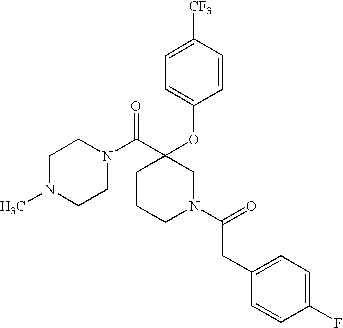
or a pharmaceutically acceptable salt, solvate, ester, or prodrug thereof to a mammal in need of such treatment.

3. A method of treating or preventing one or more diseases associated with P53, comprising administering a therapeutically effective amount of at least one compound of the following structure: 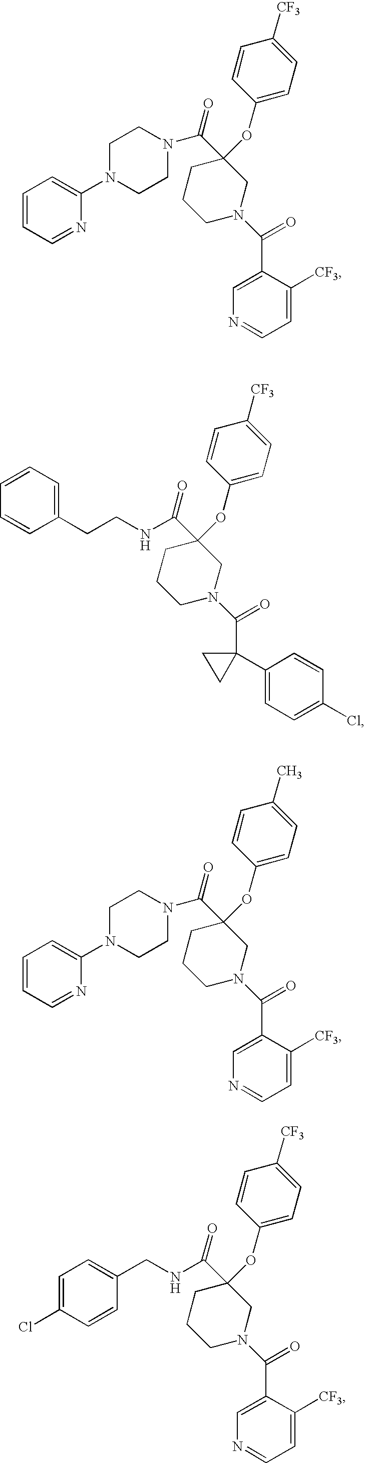 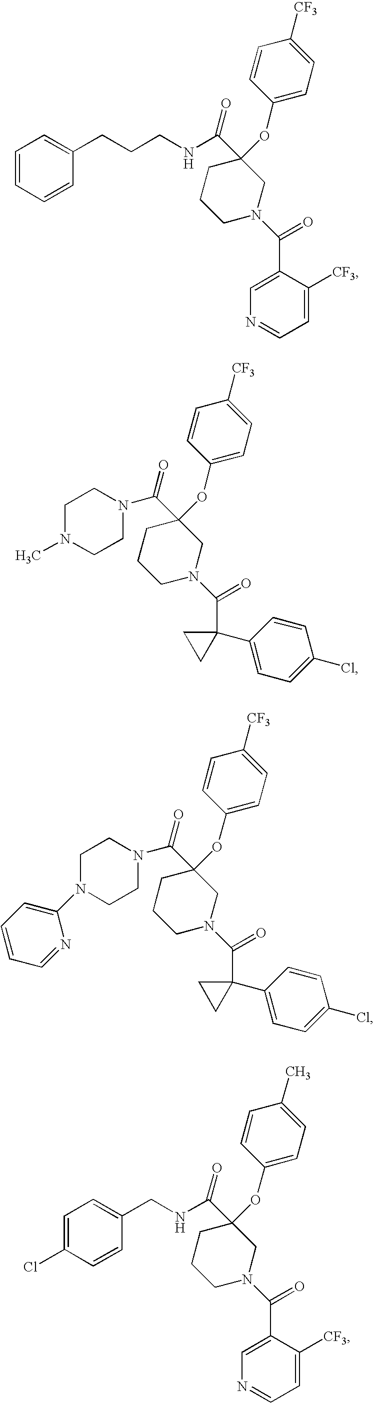 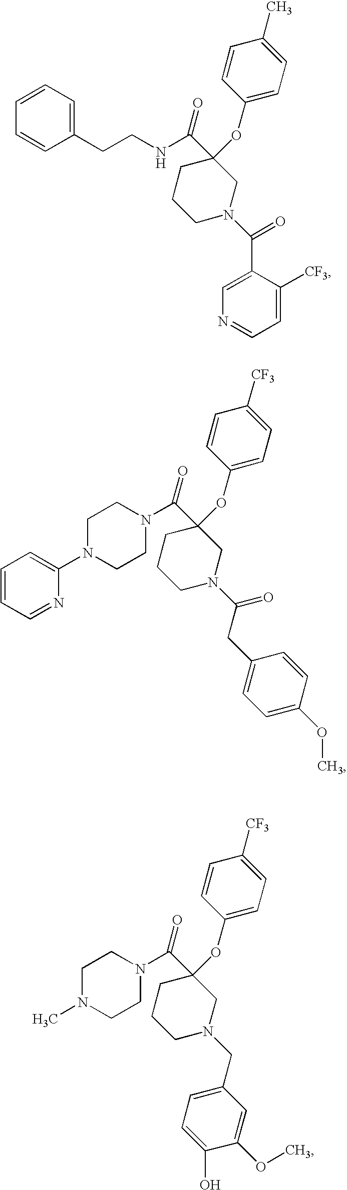 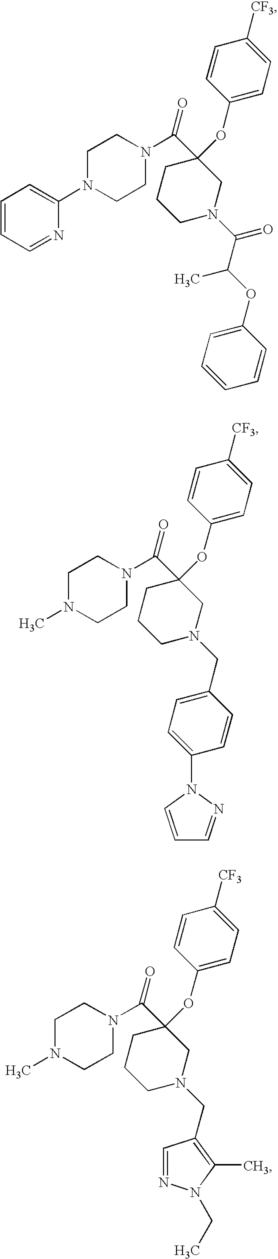 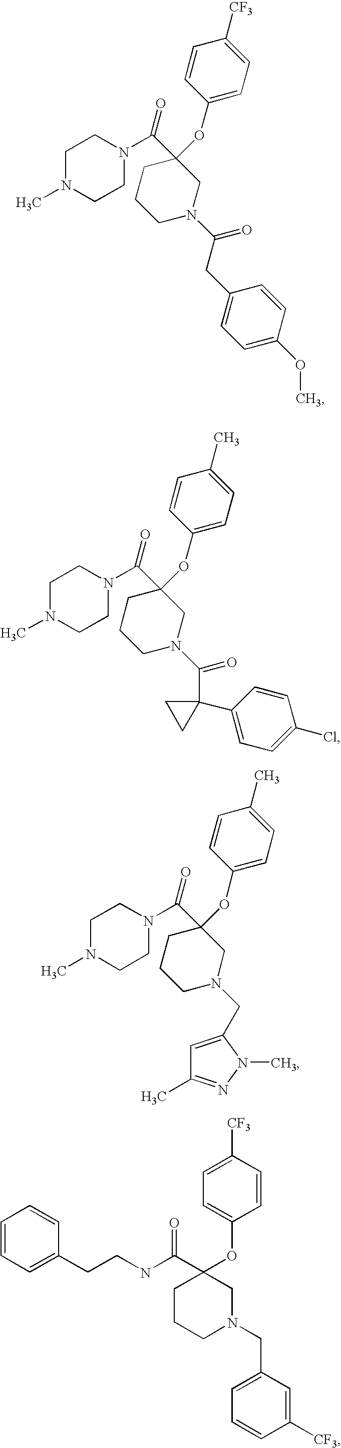 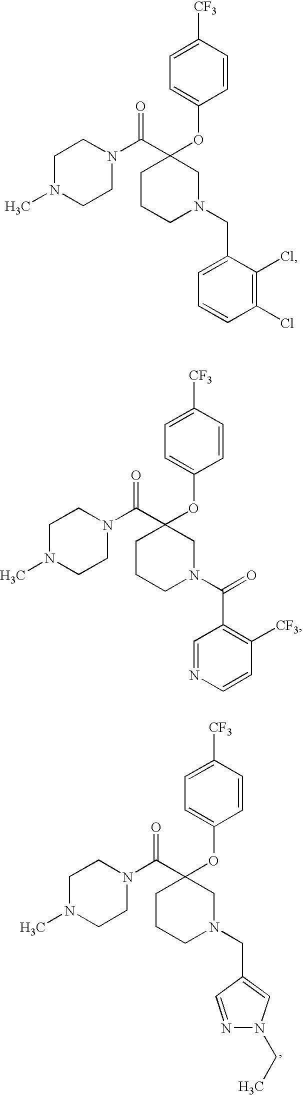 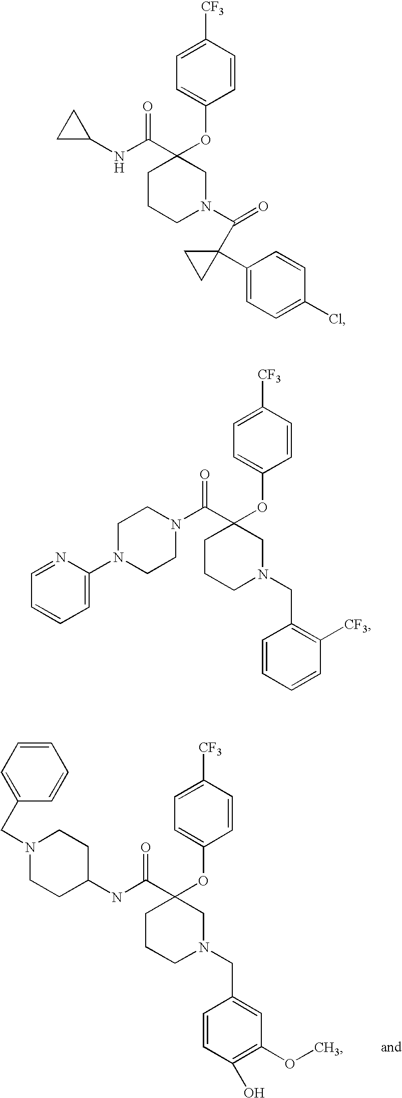 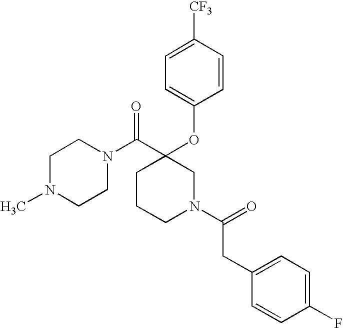
or a pharmaceutically acceptable salt, solvate, esters or prodrug to a mammal in need of such treatment.

4. A method of treating or preventing one or more diseases associated with HDM2 interacting with P53, comprising administering a therapeutically effective amount of at least one compound of the following structure: 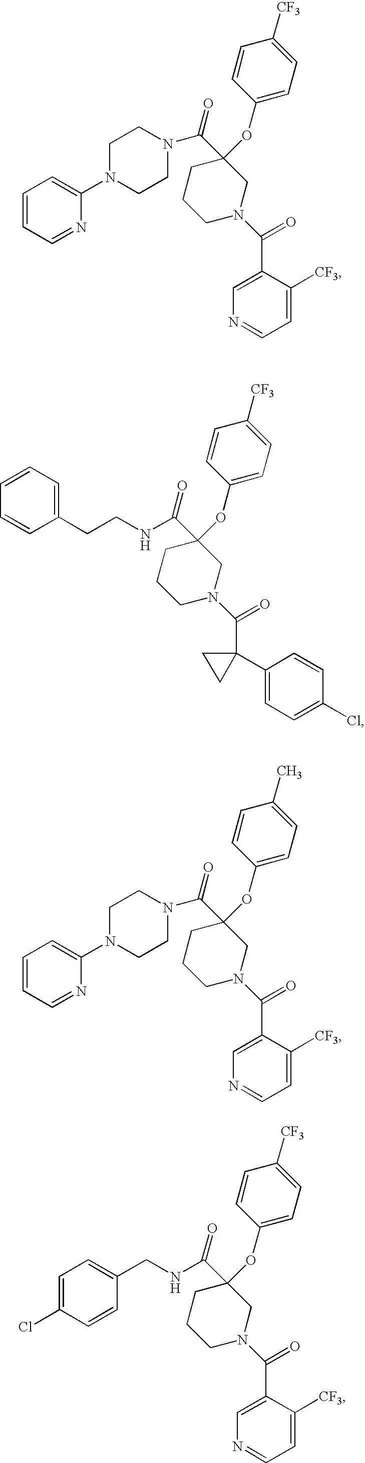 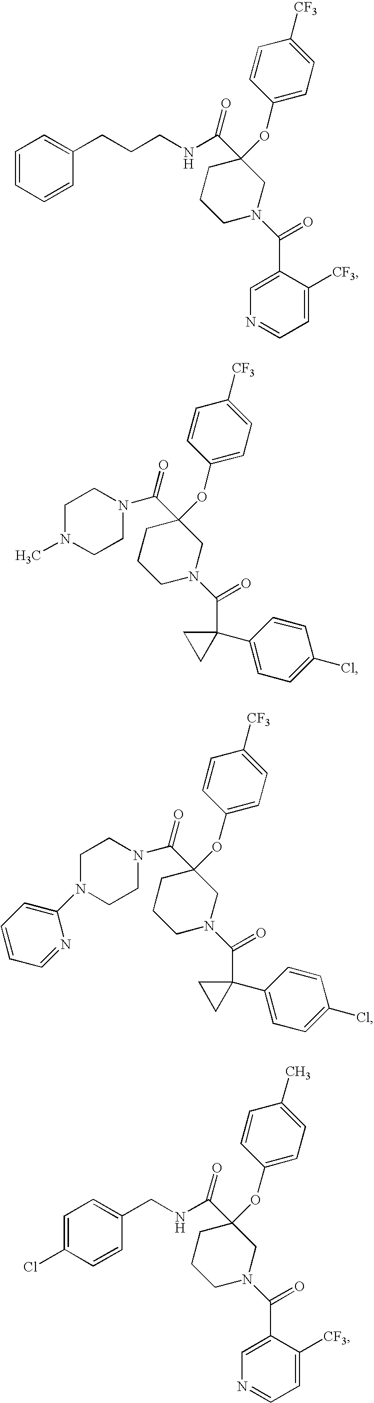 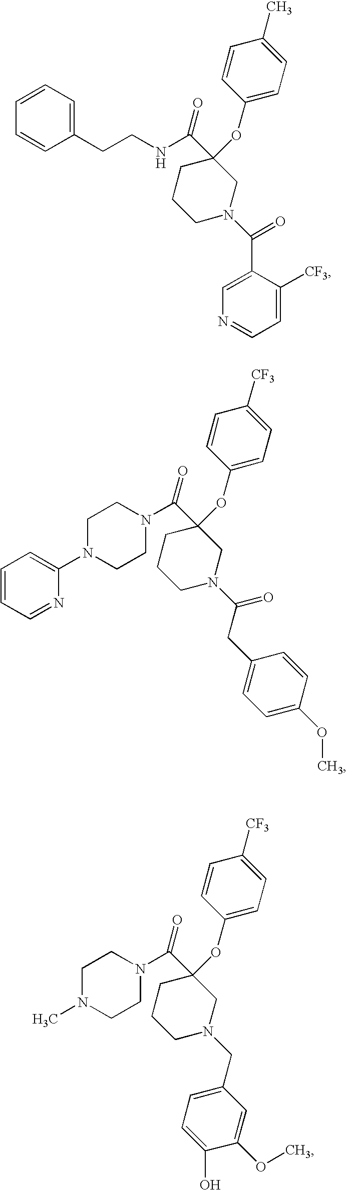 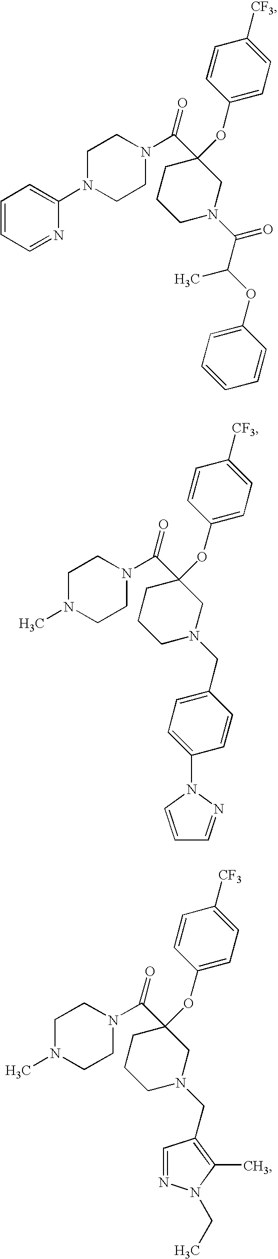 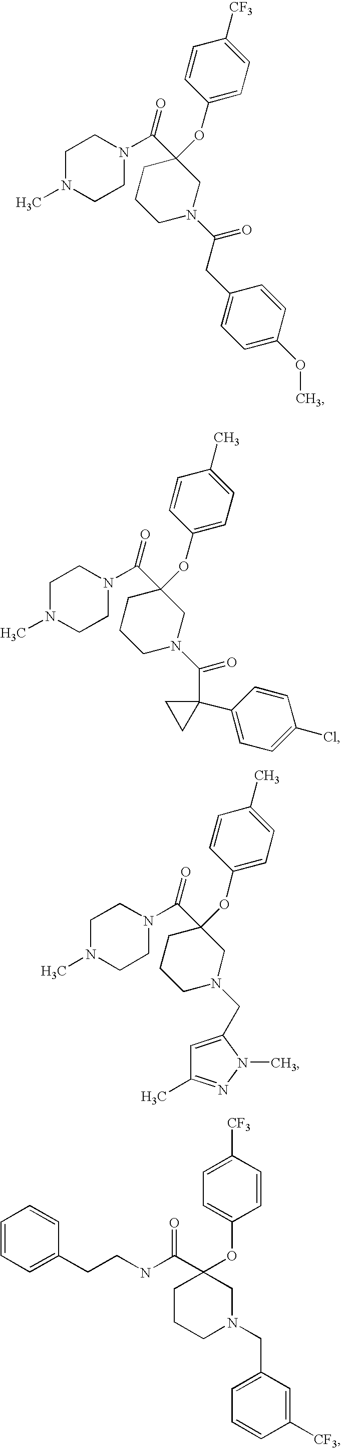 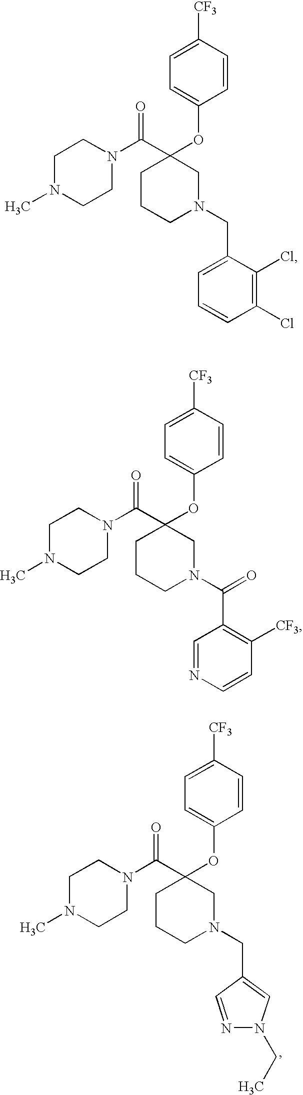 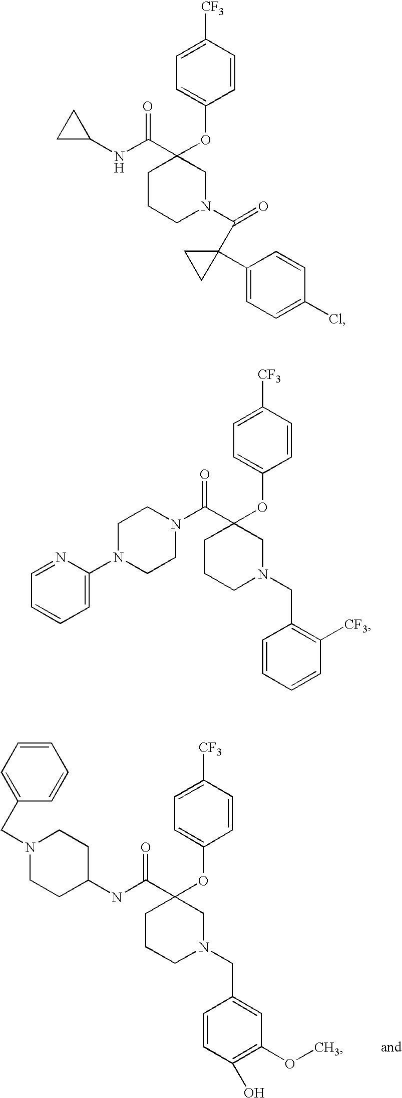 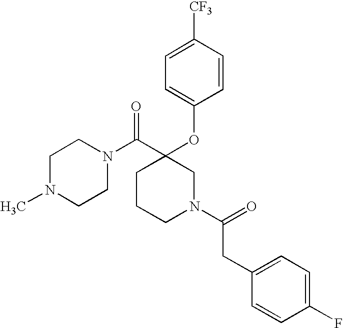
or a pharmaceutically acceptable salt, solvate, ester, or prodrug thereof to a mammal in need of such treatment.

5. A method of claim 2, comprising administering to a mammal in need of such treatment
an amount of a first compound disclosed in claim 2; and
an amount of at least one second compound, wherein said second compound is an anti-cancer agent different from the compound disclosed in claim 2;
wherein the amounts of the first compound and the second compound result in a therapeutic effect.

6. A method of claim 3, comprising administering to a mammal in need of such treatment
an amount of a first compound disclosed in claim 3; and
an amount of at least one second compound, wherein said second compound being an anti-cancer agent different from the compound disclosed in claim 3;
wherein the amounts of the first compound and the second compound result in a therapeutic effect.

7. A method of claim 4, comprising administering to a mammal in need of such treatment
an amount of a first compound disclosed in claim 4; and
an amount of at least one second compound, wherein said second compound being an anti-cancer agent different from the compound disclosed in claim 4;
wherein the amounts of the first compound and the second compound result in a therapeutic effect.

8. The method according to any of claims 2-7, wherein the disease is selected from the group consisting of:
carcinoma, including, but not limited to, of the bladder, breast, colon, rectum, endometrium, kidney, liver, lung, head and neck, esophagus, gall bladder, cervix, pancreas, prostrate, larynx, ovaries, stomach, uterus, sarcoma and thyroid cancer;
hematopoietic tumors of the lymphoid lineage, including leukemia, acute lymphocytic leukemia, chronic lymphocytic leukemia, acute lymphoblastic leukemia, B-cell lymphoma, T-cell lymphoma, Hodgkins lymphoma, non-Hodgkins lymphoma, hairy cell lymphoma, mantle cell lymphoma, myeloma, and Burkett's lymphoma;
hematopoetic tumors of myeloid lineage, including acute and chronic myelogenous leukemias, myelodysplastic syndrome and promyelocytic leukemia;
tumors of mesenchymal origin, including fibrosarcoma and rhabdomyosarcoma;
tumors of the central and peripheral nervous system, including astrocytoma, neuroblastoma, glioma, and schwannomas; and
other tumors, including melanoma, skin (non-melanomal) cancer, mesothelioma (cells), seminoma, teratocarcinoma, osteosarcoma, xenoderoma pigmentosum, keratoctanthoma, thyroid follicular cancer and Kaposi's sarcoma.

9. The method according to any of claims 2 to 7 further comprising radiation therapy, surgery, chemotherapy, biological therapy, hormone therapy, photodynamic therapy, or bone marrow transplant.

10. The method according to claims 5, 6, or 7, wherein the anti-cancer agent is selected from the group consisting of a cytotoxic agents, targeted therapeutic agents (small molecules, biologics, siRNA and microRNA) against cancer and neoplastic diseases,
anti-metabolites (such as methoxtrexate, 5-fluorouracil, gemcitabine, fludarabine, capecitabine);
alkylating agents, such as temozolomide, cyclophosphamide,
DNA interactive and DNA damaging agents, such as cisplatin, oxaliplatin, doxorubicin,
Ionizing irradiation, such as radiation therapy,
topoisomerase II inhibitors, such as etoposide, doxorubicin,
topoisomerase I inhibitors, such as irinotecan, topotecan,
tubulin interacting agents, such as paclitaxel, docetaxel, Abraxane, epothilones,
kinesin spindle protein inhibitors,
spindle checkpoint inhibitors,
Poly(ADP-ribose) polymerase (PARP) inhibitors
Matrix metalloprotease (MMP) inhibitors
Protease inhibitors, such as cathepsin D and cathepsin K inhibitors
Proteosome or ubiquitination inhibitors, such as bortezomib,
Activator of mutant P53 to restore its wild-type P53 activity
Adenoviral-P53
Bcl-2 inhibitors, such as ABT-263
Heat shock protein (HSP) modulators, such as geldanamycin and 17-AAG
Histone deacetylase (HDAC) inhibitors, such as vorinostat (SAHA),
sex hormone modulating agents,
anti-estrogens, such as tamoxifen, fulvestrant,
selective estrogen receptor modulators (SERM), such as raloxifene,
anti-androgens, such as bicalutamide, flutamide
LHRH agonists, such as leuprolide,
5α-reductase inhibitors, such as finasteride,
Cytochrome P450 C17 lysase (CYP450c17) inhibitors, such as Abiraterone
aromatase inhibitors, such as letrozole, anastrozole, exemestane,
EGFR kinase inhibitors, such as geftinib, erlotinib, laptinib
dual erbB1 and erbB2 inhibitors, such as Lapatinib
multi-targeted kinases (serine/threonine and/or tyrosine kinase) inhibitors,
ABL kinase inhibitors, imatinib and nilotinib, dasatinib
VEGFR-1, VEGFR-2, PDGFR, KDR, FLT, c-Kit, Tie2, Raf, MEK and ERK
inhibitors, such as sunitinib, sorafenib, Vandetanib, pazopanib, Axitinib,
PTK787,
Polo-like kinase inhibitors,
Aurora kinase inhibitors,
JAK inhibitor
c-MET kinase inhibitors
Cyclin-dependent kinase inhibitors, such as CDK1 and CDK2 inhibitor SCH 727965
PI3K inhibitors
mTOR inhibitors, such as Rapamycin, Temsirolimus, and RAD001 and other anti-cancer (also know as anti-neoplastic) agents include but are not limited to ara-C, adriamycin, cytoxan, Carboplatin, Uracil mustard, Clormethine, Ifosfsmide, Melphalan, Chlorambucil, Pipobroman, Triethylenemelamine, Triethylenethiophosphoramine, Busulfan, Carmustine, Lomustine, Streptozocin, Dacarbazine, Floxuridine, Cytarabine, 6-Mercaptopurine, 6-Thioguanine, Fludarabine phosphate, Pentostatine, Vinblastine, Vincristine, Vindesine, Vinorelbine, Navelbine, Bleomycin, Dactinomycin, Daunorubicin, Doxorubicin, Epirubicin, teniposide, cytarabine, pemetrexed, Idarubicin, Mithramycin, Deoxycoformycin, Mitomycin-C, L-Asparaginase, Teniposide 17α-Ethinylestradiol, Diethylstilbestrol, Testosterone, Prednisone, Fluoxymesterone, Dromostanolone propionate, Testolactone, Megestrolacetate, Methylprednisolone, Methyltestosterone, Prednisolone, Triamcinolone, Chlorotrianisene, Hydroxyprogesterone, Aminoglutethimide, Estramustine, Flutamide Medroxyprogesteroneacetate, Toremifene, goserelin, Carboplatin, Hydroxyurea, Amsacrine, Procarbazine, Mitotane, Mitoxantrone, Levamisole, Drolloxafine, Hexamethylmelamine, Bexxar, Zevalin, Trisenox, Profimer, Thiotepa, Altretamine, Doxil, Ontak, Depocyt, Aranesp, Neupogen, Neulasta, Kepivance.
Farnesyl protein transferase inhibitors, such as, SARASAR™ (4-[2-[4-[(11R)-3,10-dibromo-8-chloro-6,11-dihydro-5H-benzo[5,6]cyclohepta[1,2-b]pyridin-11-yl-]-1-piperidinyl]-2-oxoethyl]-piperidinecarboxamide, tipifarnib
interferons, such as Intron A, Peg-Intron,
anti-erbB1 antibodies, such as cetuximab, panitumumab,
anti-erbB2 antibodies, such as trastuzumab,
anti-CD52 antibodies, such as Alemtuzumab,
anti-CD20 antibodies, such as Rituximab
anti-CD33 antibodies, such as Gemtuzumab ozogamicin
anti-VEGF antibodies, such as Avastin,
TRIAL ligands, such as Lexatumumab, mapatumumab, and AMG-655 antibodies against CTLA-4, CTA1, CEA, CD5, CD19, CD22, CD30, CD44, CD44V6, CD55, CD56, EpCAM, FAP, MHCII, HGF, IL-6, MUC1, PSMA, TAL6, TAG-72, TRAILR, VEGFR, IGF-2, FGF,
anti-IGF-1R antibodies, such as SCH 717454.

11. The method of claim 1, further comprising adding a pharmaceutically acceptable carrier to the compounds disclosed in claim 1.

12. Method of targeting HDM2-P53 interaction for the treatment of diseases of a mammal through activation of P53 activities comprising administering a therapeutically effective amount of at least one compound of claim 1 or a pharmaceutically acceptable salt, solvate, ester, or prodrug thereof to a mammal in need of such treatment.

13. The method of any of claims 1-7 and 12, wherein the mammal is a human.

14. Method of protecting normal, healthy cells of a mammal from cytotoxic induced side-effects comprising administering at least one compound of claim 1 or a pharmaceutically acceptable salt, solvate, ester or prodrug thereof prior to administration of anticancer agents other than the compounds of claim 1 to a mammal carrying mutated P53.

15. The method of claim 14, wherein said other anticancer agent is paclitaxel.

16. The method of claim 12, wherein an amount of said first compound, which is a compound of claim 1, or a pharmaceutically acceptable salt, solvate, or ester thereof can be administered simultaneously, consecutively, or sequentially with an amount of at least one second compound, the second compound being an anti-cancer agent different from the compound of claim 1.
